(19) United States
(12) Patent Application Publication
(10) Pub. No.: US 2023/0310652 A1
(43) Pub. Date: Oct. 5, 2023

(54) VLP FOR THE TREATMENT OF LEUKODYSTROPHIES

(71) Applicant: NEUWAY Pharma GmbH, Bonn (DE)

(72) Inventors: Victoria DEMINA, Bonn (DE); Oliver ERNST, Bonn (DE); Anke BONSE, Bonn (DE); Markus STAPF, Bonn (DE)

(73) Assignee: NEUWAY Pharma GmbH, Bonn (DE)

(21) Appl. No.: 17/926,678

(22) PCT Filed: May 19, 2021
(86) PCT No.: PCT/EP2021/063375

§ 371 Date: Nov. 21, 2022

(30) Foreign Application Priority Data
May 22, 2020 (WO) PCT/EP2020/064324

(51) Int. Cl. A61K 48/00; C12N 15/86; C12N 9/80; C12N 9/24

(52) U.S. Cl. CPC A61K 48/005; C12N 9/2402; C12N 9/80; C12N 15/86; C12Y 302/01046; C12Y 305/01015; C12N 2710/22023; C12N 2710/22042

(57) ABSTRACT

The invention relates to virus like particles (VLP) associated with an enzyme abnormally expressed in particular leukodystrophies or an expression vector encoding the enzyme or an mRNA encoding the enzyme or a combination thereof which are used in a method for the treatment of the particular leukodystrophies in a subject in the need thereof, preferably a human. The invention also relates to a pharmaceutical composition for use in a method for the treatment of the particular leukodystrophies, to an expression vector encoding the abnormally expressed enzyme and to a method of associating a VLP with the enzyme, an expression vector encoding the enzyme or an mRNA encoding the enzyme or a combination thereof.

### CROSS REFERENCE TO RELATED APPLICATIONS

This application is a National Stage of International Application No. PCT/EP2021/063375 filed on May 19, 2021, claiming priority based on International Application No. PCT/EP2020/064324 filed on May 22, 2020.

### FIELD OF THE INVENTION

The invention relates to virus like particles (VLP) associated with an enzyme abnormally expressed in particular leukodystrophies or an expression vector encoding the enzyme or an mRNA encoding the enzyme or a combination thereof which are used in a method for the treatment of the particular leukodystrophies in a subject in the need thereof, preferably a human. The invention also relates to a pharmaceutical composition for use in a method for the treatment of the particular leukodystrophies, to an expression vector encoding the abnormally expressed enzyme and to a method of associating a VLP with the enzyme, an expression vector encoding the enzyme, an mRNA encoding the enzyme or a combination thereof.

### BACKGROUND OF THE INVENTION

Leukodystrophies are a group of rare, progressive, metabolic, genetic diseases that affect the brain, spinal cord and often the peripheral nerves. Each type of leukodystrophy is caused by a specific gene abnormality that leads to abnormal development or destruction of the white matter (myelin sheath) of the brain. Each type of leukodystrophy affects a different part of the myelin sheath, leading to a range of neurological problems.

One example of a leukodystrophy is Canavan disease (CD). CD is a rare inherited neurological disorder characterized by spongy degeneration of the brain and spinal cord (central nervous system). Physical symptoms that appear in early infancy may include progressive mental decline accompanied by the loss of muscle tone, poor head control, an abnormally large head (macrocephaly), and/or irritability. Physical symptoms appear in early infancy and usually progress rapidly.

CD is caused by a defective ASPA gene which is responsible for the production of the enzyme aspartoacylase. Decreased aspartoacylase activity prevents the normal breakdown of N-acetyl aspartate, wherein the accumulation of N-acetyl aspartate, or lack of its further metabolism interferes with growth of the myelin sheath of the nerve fibres of the brain.

Another example of a leukodystrophy is Krabbe disease. Krabbe disease, also known as globoid cell leukodystrophy, is an autosomal recessive lipid storage disorder caused by mutations in the GALC gene located on chromosome 14 (14q31), which is inherited in an autosomal recessive manner. Mutations in the GALC gene cause a deficiency of the lysosomal enzyme galactocerebrosidase, which is necessary for the breakdown (metabolism) of the sphingolipids galactosylceramide and psychosine (galactosylsphingosine). Failure to break down these sphingolipids results in degeneration of the myelin sheath surrounding nerves in the brain (demyelination). Characteristic globoid cells appear in affected areas of the brain. This metabolic disorder is characterized by progressive neurological dysfunction with irritability, developmental regression, abnormal body tone, seizures and peripheral neuropathy.

Gene therapeutic approaches based on recombinant adenoassociated viruses have been discussed in the prior art for the treatment of leukodystrophies like Canavan disease and Krabbe disease.

However, safety concerns arise when, viral vectors are administered.

Hence, a need exists for safe and effective therapies for the treatment of leukodystrophies, such as Canavan disease and Krabbe disease. It is thus an object of the present invention to provide means for novel therapies for the treatment of leukodystrophies like Canavan disease and Krabbe disease, which overcome previous constraints.

### SUMMARY OF THE INVENTION

The inventors have found that virus-like particles (VLP), more specifically VLP of the JC virus, associated with an enzyme abnormally expressed in a particular leukodystrophy or an expression vector encoding such an enzyme or an mRNA encoding such an enzyme or a combination thereof are particularly well suited for the treatment of the particular leukodystrophy. The VLP according to the invention can be used for the efficacious delivery of functional enzymes or plasmids encoding such enzymes or mRNA encoding such enzymes or a combination thereof into the CNS in a patient in the need thereof. To achieve this, according to the invention, the VLP is associated with an enzyme or an expression vector encoding an enzyme or an mRNA encoding an enzyme or a combination thereof, whose absence or reduced activity is responsible for the leukodystrophy to be treated.

In particular, the inventors have found that virus-like particles (VLP), more specifically VLP of the JC virus, associated with aspartoacylase or an expression vector encoding aspartoacylase or an mRNA encoding aspartoacylase or a combination thereof are particularly well suited for the treatment of Canavan disease. The VLP according to the invention can be used for the efficacious delivery of functional aspartoacylase or plasmids encoding aspartoacylase or mRNA encoding aspartoacylase or a combination thereof into the CNS in a patient in the need thereof. To achieve this, according to the invention, the VLP is associated with aspartoacylase or an expression vector encoding aspartoacylase or an mRNA encoding aspartoacylase or a combination thereof, whose absence or reduced activity is responsible for Canavan disease.

Further, the inventors have found that virus-like particles (VLP), more specifically VLP of the JC virus, associated with galactocerebrosidase or an expression vector encoding galactocerebrosidase or an mRNA encoding galactocerebrosidase or a combination thereof are particularly well suited for the treatment of Krabbe disease. The VLP according to the invention can be used for the efficacious delivery of functional galactocerebrosidase or plasmids encoding galactocerebrosidase or mRNA encoding galactocerebrosidase or a combination thereof into the CNS in a patient in the need thereof. To achieve this, according to the invention, the VLP is associated with galactocerebrosidase or an expression vector encoding galactocerebrosidase or an mRNA encoding galactocerebrosidase or a combination thereof, whose absence or reduced activity is responsible for Krabbe disease.

In keeping with this, in its broadest sense an “enzyme” in accordance with the invention relates to a polypeptide having aspartoacylase or galactocerebrosidase activity, respectively.

The VLP according to the invention can effectively cross the blood brain barrier (BBB), advantageously even the physiologically intact BBB. Hence the VLP associated with aspartoacylase or galactocerebrosidase, respectively, or an expression vector encoding the respective enzyme or mRNA encoding the respective enzyme or a combination thereof can effectively deliver the enzyme or the expression vector encoding the enzyme or the mRNA encoding the respective enzyme or the combination thereof into the CNS.

The inventors found ASPA and GALC mRNA, respectively, in different mouse and human cell lines and in mouse brains after administration of the VLP according to the invention. They also found the permeation through the blood-brain-barrier (BBB) with VLP in vitro associated with aspartoacylase (ASPA) or galactocerebrosidase (GALC) according to the invention in an artificial BBB model. Thereby, it can be concluded that with the administration of VLP according to the invention the respective enzyme activity in the CNS can be enhanced. The enhanced enzyme activity enables the effective treatment of Canavan disease and Krabbe disease, respectively; if the enzyme is aspartoacylase (ASPA) and galactocerebrosidase (GALC), respectively.

Surprisingly, it has been found that the VLP according to the invention target specifically the CNS, i.e. they do not equally distribute over the body after having been administered to the patient, e.g. intravenously. That means that a larger proportion of VLP according to the invention is found in the CNS than outside the CNS.

The VLP according to the invention particularly target astrocytes, oligodendrocytes, neurons and/or microglia. A specifically preferred target of the VLP of the invention are oligodendrocytes.

The VLP according to the invention are also stable and homogeneous which is especially important for clinical use because it allows for a better quality management and standardization of the drug product.

Thus, it has been shown by the inventors, that VLP, in particular VLP of the JC virus, associated with aspartoacylase and galactocerebrosidase, respectively, or an expression vector encoding the respective enzyme or an mRNA encoding the respective enzyme or a combination thereof can be used to provide an efficacious and safe treatment of Canavan disease and Krabbe disease, respectively.

### BRIEF DESCRIPTION OF THE FIGURES

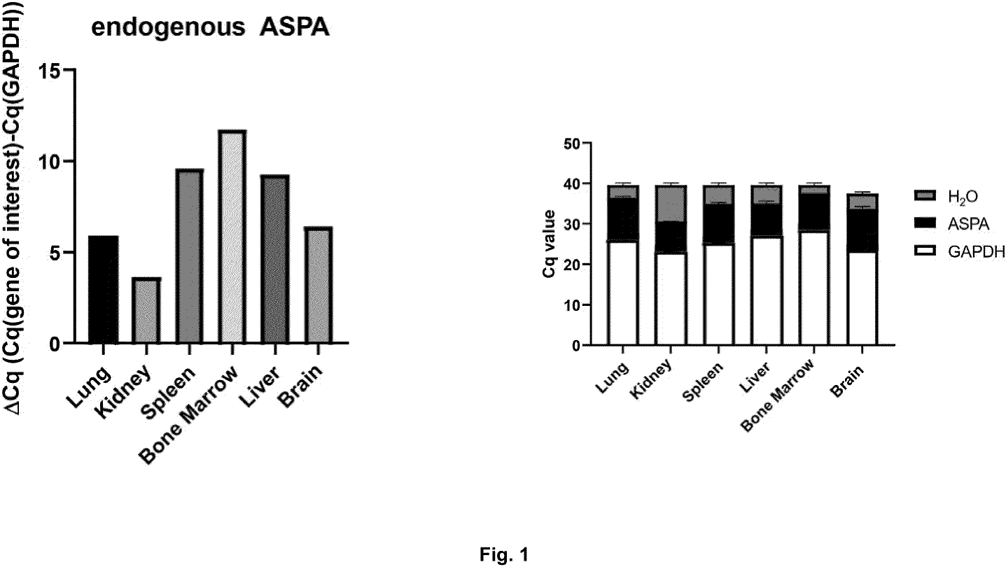

### FIG. 1 Detection of Endogenous ASPA Expression in Mouse Organs

Left panel shows detection of endogenous, i.e. murine ASPA expression in the different mouse organs with mouse specific primers. Right panel shows detection of endogenous, i.e. murine ASPA expression in the different mouse organs with primers specific for human ASPA (hASPA).

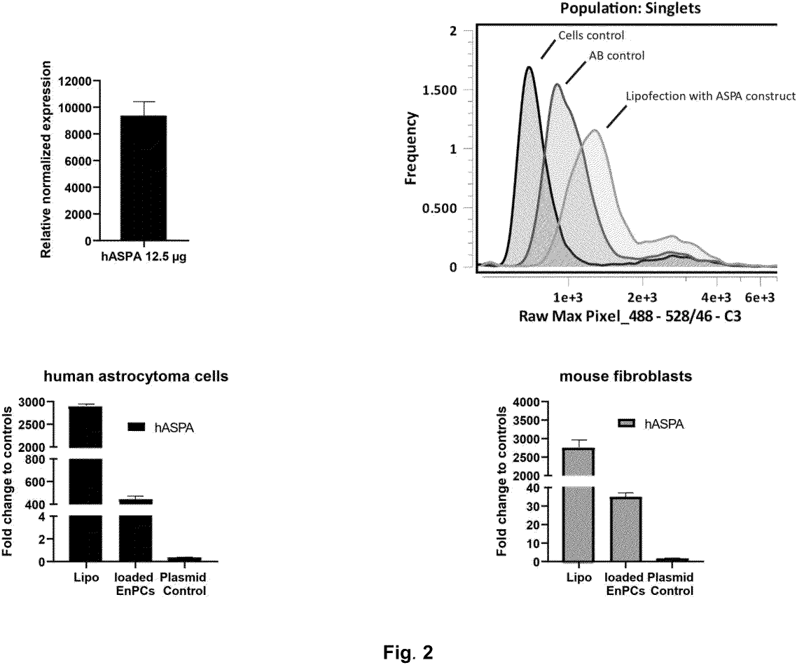

### FIG. 2 hASPA Expression

Upper left panel shows hASPA expression (mRNA) in human astrocytoma cells after incubation with encapsulated hASPA, i.e. VLP packed with a hASPA-encoding plasmid. Upper right panel shows hASPA protein expression (by FACS analysis) in human astrocytoma cells after lipofection with a hASPA-encoding plasmid. Lower left panel shows hASPA expression (mRNA) in human astrocytoma cells after lipofection with a hASPA-encoding plasmid (“Lipo”) or incubation with encapsulated hASPA, i.e. VLP packed with a hASPA-encoding plasmid (“loaded EnPCs”). Lower right panel shows hASPA expression (mRNA) in mouse fibroblasts after lipofection with a hASPA-encoding plasmid (“Lipo”) or incubation with encapsulated hASPA, i.e. VLP packed with a hASPA-encoding plasmid (“loaded EnPCs”).

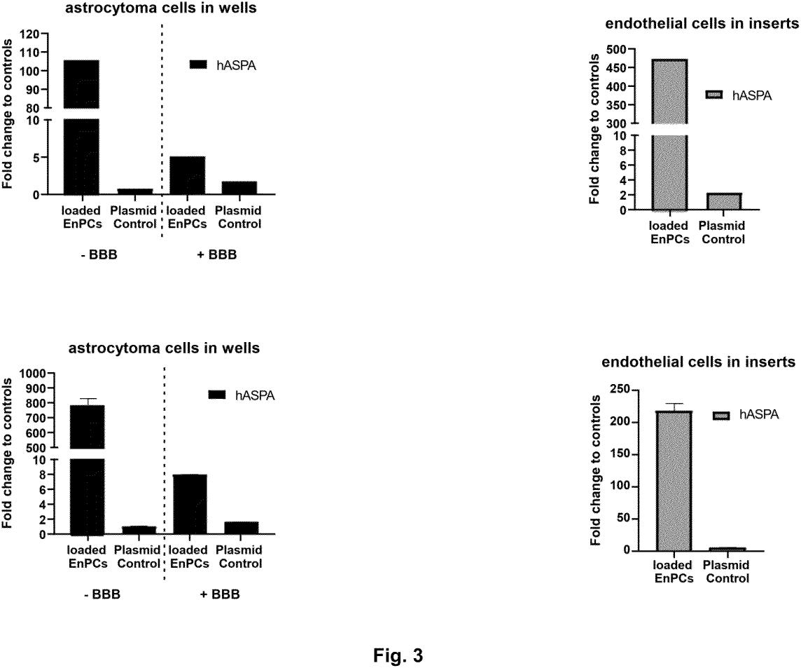

### FIG. 3 Permeation of VLP Associated With hASPA in an in Vitro BBB Model

Upper left panel shows hASPA expression (mRNA) in human astrocytoma cells after co-culture of the human astrocytoma cells in wells with (+ BBB) and without (- BBB) HBEC-5i endothelial cells seeded into inserts in the wells. VLP packed with a hASPA-encoding plasmid (“loaded EnPCs”) or the hASPA-encoding plasmid alone (“Plasmid control”) were added to the inserts and inserts were transferred to wells with fresh medium after 24 hours for another 48 h. Cell pellets were harvested after 72 h.

Upper right panel shows hASPA expression (mRNA) in HBEC-5i endothelial cells after co-culture of human astrocytoma cells in wells with the HBEC-5i endothelial cells seeded into inserts in the wells. VLP packed with a hASPA-encoding plasmid (“loaded EnPCs”) or the hASPA-encoding plasmid alone (“Plasmid control”) were added to the inserts and inserts were transferred to wells with fresh medium after 24 hours for another 48 h. Cell pellets were harvested after 72 h.

Lower left panel shows hASPA expression (mRNA) in human astrocytoma cells after co-culture of the human astrocytoma cells in wells with (+ BBB) and without (- BBB) HBEC-5i endothelial cells seeded into inserts in the wells. VLP packed with a hASPA-encoding plasmid (“loaded EnPCs”) or the hASPA-encoding plasmid alone (“Plasmid control”) were added to the inserts and inserts were transferred to wells with fresh medium after 48 hours for another 24 h. Cell pellets were harvested after 72 h.

Lower right panel shows hASPA expression (mRNA) in HBEC-5i endothelial cells after co-culture of human astrocytoma cells in wells with the HBEC-5i endothelial cells seeded into inserts in the wells. VLP packed with a hASPA-encoding plasmid (“loaded EnPCs”) or the hASPA-encoding plasmid alone (“Plasmid control”) were added to the inserts and inserts were transferred to wells with fresh medium after 48 h for another 24 h. Cell pellets were harvested after 72 h.

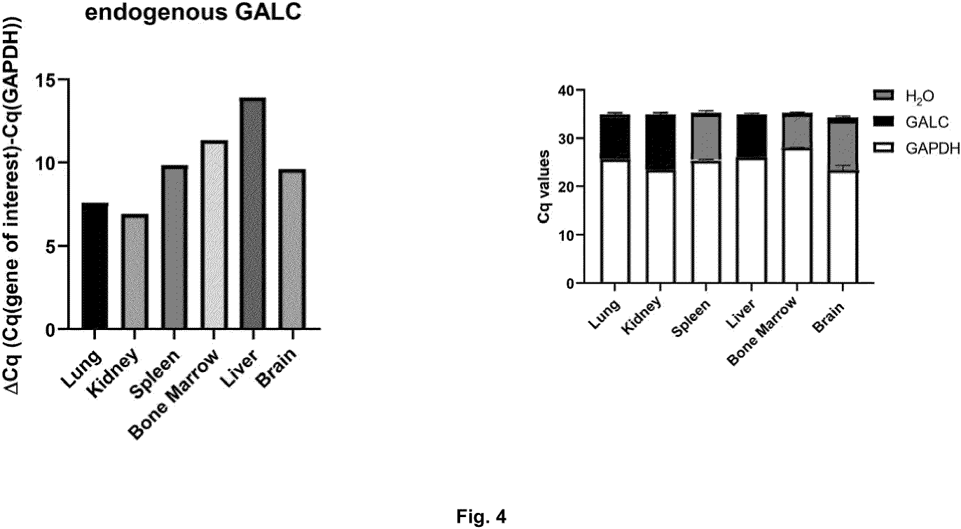

### FIG. 4 Detection of Endogenous GALC Expression in Mouse Organs

Left panel shows detection of endogenous, i.e. murine GALC expression in the different mouse organs with mouse specific primers. Right panel shows detection of endogenous, i.e. murine GALC expression in the different mouse organs with primers specific for human GALC (hGALC).

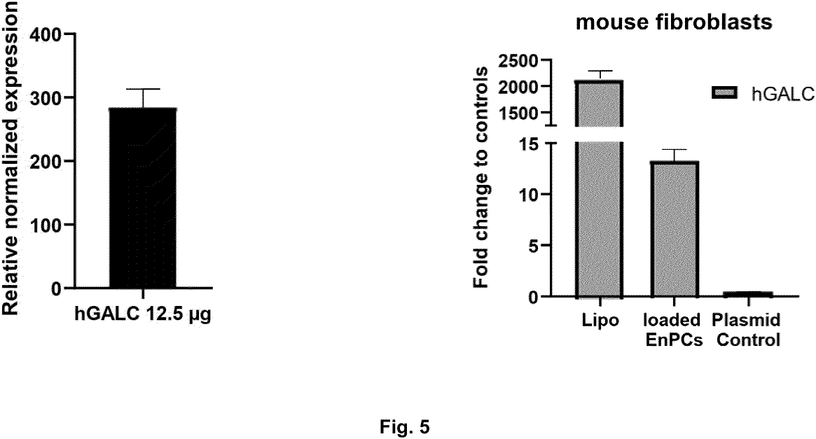

### FIG. 5 hGALC Expression

Left panel shows hGALC expression (mRNA) in human astrocytoma cells after incubation with encapsulated hGALC, i.e. VLP packed with a hGALC-encoding plasmid. Left panel shows hGALC expression (mRNA) in mouse fibroblasts after lipofection with a hGALC-encoding plasmid (“Lipo”) or incubation with encapsulated hGALC, i.e. VLP packed with a hGALC-encoding plasmid (“loaded EnPCs”).

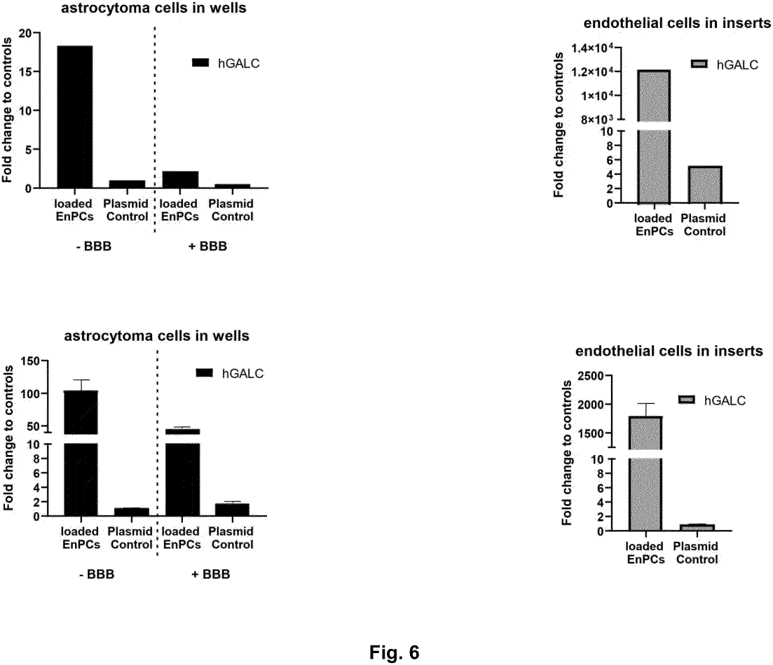

### FIG. 6 Permeation of VLP Associated With hGALC in an in Vitro BBB Model

Upper left panel shows hGALC expression (mRNA) in human astrocytoma cells after co-culture of the human astrocytoma cells in wells with (+ BBB) and without (- BBB) HBEC-5i endothelial cells seeded into inserts in the wells. VLP packed with a hGALC-encoding plasmid (“loaded EnPCs”) or the hGALC-encoding plasmid alone (“Plasmid control”) were added to the inserts and inserts were transferred to wells with fresh medium after 24 hours for another 48 h. Cell pellets were harvested after 72 h.

Upper right panel shows hGALC expression (mRNA) in HBEC-5i endothelial cells after co-culture of human astrocytoma cells in wells with the HBEC-5i endothelial cells seeded into inserts in the wells. VLP packed with a hGALC-encoding plasmid (“loaded EnPCs”) or the hGALC-encoding plasmid alone (“Plasmid control”) were added to the inserts and inserts were transferred to wells with fresh medium after 24 hours for another 48 h. Cell pellets were harvested after 72 h.

Lower left panel shows hGALC expression (mRNA) in human astrocytoma cells after co-culture of the human astrocytoma cells in wells with (+ BBB) and without (- BBB) HBEC-5i endothelial cells seeded into inserts in the wells. VLP packed with a hGALC-encoding plasmid (“loaded EnPCs”) or the hGALC-encoding plasmid alone (“Plasmid control”) were added to the inserts and inserts were transferred to wells with fresh medium after 48 hours for another 24 h. Cell pellets were harvested after 72 h.

Lower right panel shows hGALC expression (mRNA) in HBEC-5i endothelial cells after co-culture of human astrocytoma cells in wells with the HBEC-5i endothelial cells seeded into inserts in the wells. VLP packed with a hGALC-encoding plasmid (“loaded EnPCs”) or the hGALC-encoding plasmid alone (“Plasmid control”) were added to the inserts and inserts were transferred to wells with target cells in fresh medium after 48 h for another 24 h. Cell pellets were harvested after 72 h.

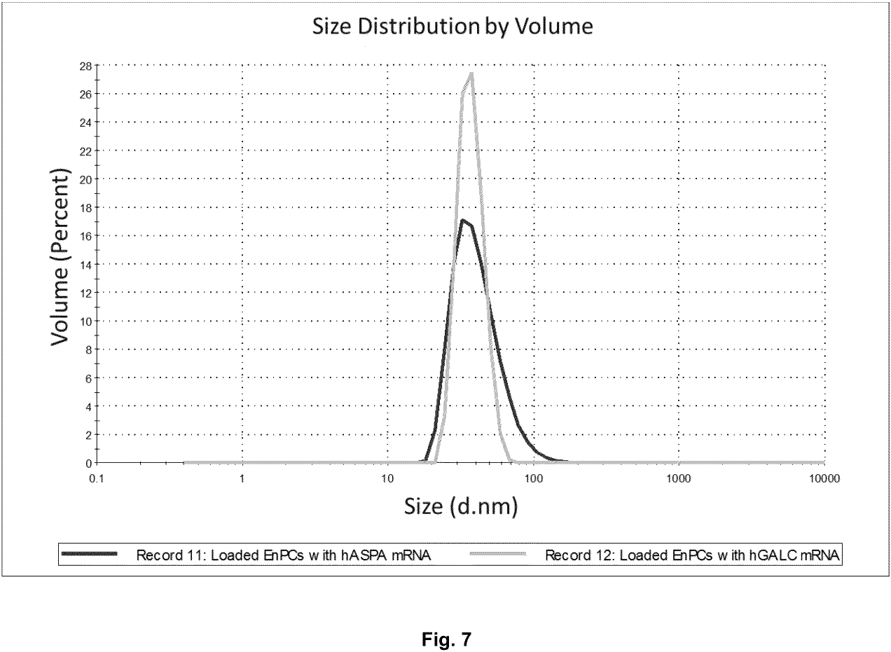

### FIG. 7 DLS Analyses of VLP Associated With hASPA or hGALC mRNA

The diagram shows DLS analyses of VLP associated with hASPA mRNA (black line; “Loaded EnPCs with hASPA mRNA”) or hGALC mRNA (grey line; “Loaded EnPCs with hGALC mRNA”).

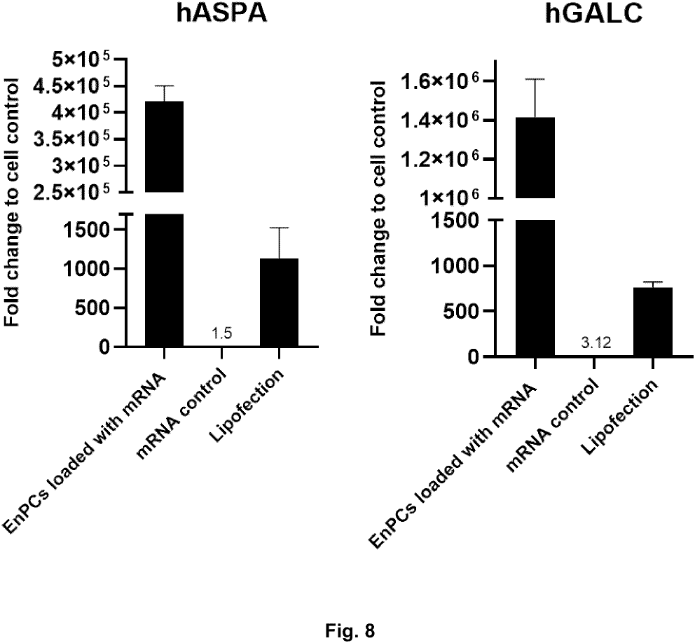

### FIG. 8 hASPA and hGALC Expression in Vitro (mRNA as Cargo)

Left panel shows hASPA expression (mRNA; qPCR analyses of two technical replicates) in human astrocytoma cells after incubation with VLP packed with a hASPA-encoding mRNA (“EnPCs loaded with mRNA”), incubation with hASPA-encoding mRNA (“mRNA control”) or lipofection with hASPA-encoding mRNA (“Lipofection”). Shown is mean with SEM.

Right panel shows hGALC expression (mRNA; qPCR analyses of two technical replicates) in human astrocytoma cells after incubation with VLP packed with a hGALC-encoding mRNA (“EnPCs loaded with mRNA”), incubation with hGALC-encoding mRNA (“mRNA control”) or lipofection with hGALC-encoding mRNA (“Lipofection”). Shown is mean with SEM.

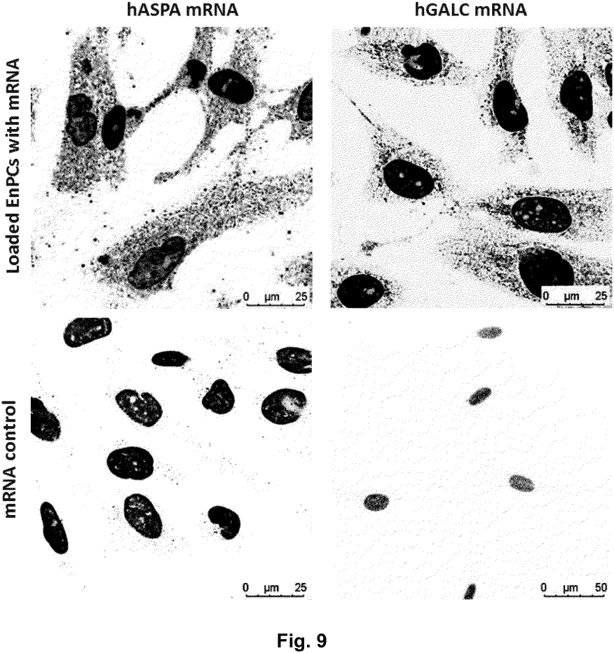

### FIG. 9 Immuncytochemistry of VLP Associated With hASPA or hGALC mRNA

Upper left panel shows human astrocytoma cells after incubation with VLP packed with a hASPA-encoding mRNA (“Loadad EnPCs with mRNA”) and staining with an anti-hASPA antibody. Lower left panel shows human astrocytoma cells after incubation with hASPA-encoding mRNA (“mRNA control”) and staining with an anti-hASPA antibody.

Upper right panel shows human astrocytoma cells after incubation with VLP packed with a hGALC-encoding mRNA (“Loadad EnPCs with mRNA”) and staining with an anti-hGALC antibody. Lower right panel shows human astrocytoma cells after incubation with hGALC-encoding mRNA (“mRNA control”) and staining with an anti-hGALC antibody.

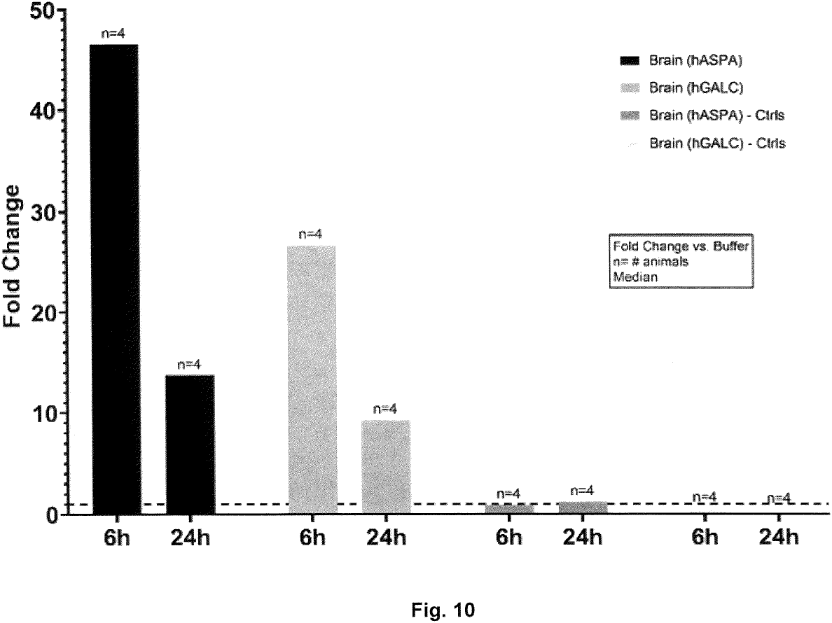

### FIG. 10 hASPA and hGALC Expression in Vivo (mRNA as Cargo)

The diagram shows mRNA expression (qPCR analyses of two technical replicates) in lysates of mice brains after injection with VLP associated with hASPA mRNA (columns 1 and 2 from left to right, “Brain (hASPA)”), injection with VLP associated with hGALC mRNA (columns 3 and 4 from left to right; “Brain (hGALC)”), injection with hASPA mRNA (columns 5 and 6 from left to right; “Brain (hASPA) - Ctrls”) or injection with hGALC mRNA (columns 7 and 8 from left to right; “Brain (hGALC) - Ctrls”) after 6 and 24 h, respectively. The number “n” depicts the number of animals. Shown is median.

### DETAILED DESCRIPTION OF THE INVENTION

In a first aspect of the invention, the invention relates to VLP associated with aspartoacylase or an expression vector encoding aspartoacylase or an mRNA encoding aspartoacylase or a combination thereof for use in a method for the treatment of Canavan disease in a subject, in particular a human. Hence the invention also relates to a method of treating a human suffering from Canavan disease with a VLP associated with aspartoacylase or an expression vector encoding aspartoacylase or an mRNA encoding aspartoacylase or a combination thereof.

In a related aspect of the invention, the invention relates to VLP associated with galactocerebrosidase or an expression vector encoding galactocerebrosidase or an mRNA encoding galactocerebrosidase or a combination thereof for use in a method for the treatment of Krabbe disease in a subject, in particular a human. Hence the invention also relates to a method of treating a human suffering from Krabbe disease with a VLP associated with galactocerebrosidase or an expression vector encoding galactocerebrosidase or an mRNA encoding galactocerebrosidase or a combination thereof.

“A combination thereof” or “the combination thereof” preferably refers to a combination of enzyme and expression vector, a combination of enzyme an mRNA, a combination of expression vector and mRNA or a combination of enzyme, expression vector an mRNA.

VLP as such, i.e. not associated with a cargo, do not comprise any genetic material because they are only built up of proteins and are otherwise “empty”. VLP according to the present invention are associated with aspartoacylase or galactocerebrosidase, respectively, or an expression vector encoding the respective enzyme or an mRNA encoding the respective enzyme or a combination thereof. In a preferred embodiment, the VLP according to the present invention do not comprise viral genetic material encoding a viral protein. In this embodiment, the VLP may comprise a viral regulatory element as viral genetic material.

Viral genetic material comprises viral genetic material encoding a viral protein and viral regulatory elements as viral genetic material. Viral genetic material is derived from the nucleic acid of a virus, i.e. at least 70 % identical to a viral RNA or DNA. Preferably, the VLP according to the invention do not comprise any viral genetic material.

Thus, preferably, in case the VLP according to the invention comprise RNA or DNA, the RNA or DNA is not derived from a virus, i.e. the RNA or DNA is not viral genetic material, in particular the RNA or DNA does not encode a viral protein. It is particularly preferred that the RNA or DNA does not encode a viral protein and does not comprise a viral regulatory element.

In a preferred embodiment, the VLP according to the invention is associated only with an expression vector or an mRNA which only encodes aspartoacylase or galactocerebrosidase, respectively, i.e. the expression vector or the mRNA does not encode any other protein. In a preferred embodiment, the expression vector or the mRNA does not encode a viral protein. It is particularly preferred that the expression vector or the mRNA does not encode a viral protein and does not comprise a viral regulatory element.

Preferably, the subject is an animal or a human being, preferably a human being.

The VLP according to the invention, advantageously, enable the delivery of aspartoacylase or galactocerebrosidase, respectively, or the expression vector encoding the respective enzyme or the mRNA encoding the respective enzyme or a combination thereof to a site of interest (“target”), preferably in a human. Preferably, the delivery is selective for the target, i.e. a higher proportion of the respective enzyme or the expression vector encoding said enzyme or the mRNA encoding said enzyme or the combination thereof is delivered to the target than to other sites of the body or organ.

The target is preferably the CNS. CNS refers to the spinal cord and the brain, in particular to the brain. The term “brain” includes anatomical parts thereof, such as frontal lobe, parietal lobe, temporal lobe, occipital lobe, and cerebellum.

It is particularly advantageous if aspartoacylase or galactocerebrosidase, respectively, or the expression vector encoding the respective enzyme or the mRNA encoding the respective enzyme or a combination thereof is delivered to and/or into a target cell, preferably a target cell in the CNS, in particular an astrocyte, an oligodendrocyte, a neuron and/or microglia. In a particularly preferred embodiment, the target cell is an oligodendrocyte. Hence, aspartoacylase or galactocerebrosidase, respectively, or the expression vector or the mRNA or the combination thereof preferably enters astrocytes, oligodendrocytes, microglia or neurons, in particular oligodendrocytes.

It is particularly advantageous when the target cell is contacted with an effective amount of aspartoacylase or galactocerebrosidase, respectively. An effective amount of the respective enzyme means that the amount is sufficient to enhance the activity of the enzyme whose absence causes the respective leukodystrophy.

Enzyme activity in the subject is usually measured in in vitro probes, typically in probes derived from solid tissues, leukocytes, fibroblasts, cultured amniotic fluid cells, serum, amniotic fluid, urine or tear drops to which a substrate is added. For example, for aspartoacylase a typical substrate is N-acetyl-L-aspartic acid (NAA), which can be directly measured as [14C]-radiolabeled NAA (Madhavarao et al., Anal. Biochem. 2002; 308: 314-319) or indirect in a coupled reaction in which the conversion of NADH to NAD+ is measured photometrically (Matalon et al., Am. J. Med. Genet. 1988; 29: 463-471). Similarly, for galactocerebrosidase a typical substrate is the fluorogenic substrate 4-methylumbelliferone-beta-galactopyranoside (4-MU-beta-D-galactosidase, MUGAL (Martino et al., Clin. Chem. 2009; 55: 541-548).

In a particularly preferred embodiment, the VLP according to the invention cross the blood-brain barrier, preferably the physiologically intact blood-brain barrier, to enter the CNS together with aspartoacylase or galactocerebrosidase, respectively, or the expression vector or the mRNA or a combination thereof. In other words, the VLP according to the invention preferably cross the BBB without a prior increase of the permeability of the BBB. The VLP according to the invention are capable of crossing the physiologically intact BBB.

The crossing of the BBB is especially advantageous if the target is the CNS, in particular if the target cell is an astrocyte, an oligodendrocyte, neuron and/or microglia. Hence, the VLP according to the invention can be used in a method of treatment of Canavan disease and Krabbe disease, respectively, wherein the method does not comprise a prior step of increasing the permeability of the BBB of the subject to be treated. The VLP according to the invention, preferably, is administered to a patient who has not received before administration any chemical or physical treatment for impairing or disrupting the BBB.

In a further embodiment, thus, the VLP associated with aspartoacylase or galactocerebrosidase, respectively, or an expression vector encoding the respective enzyme or an mRNA encoding the respective enzyme or a combination thereof or the pharmaceutical composition comprising the VLP according to the invention are free of any additive that can impact the permeability of the BBB.

The integrity of the BBB in vitro may be measured by known methods, for example by relative transendothelial electrical resistance measurement (TEER) (Rempe et al., Biochem Bioph Res Comm 2011, 406 (1): 64-69). Many in vitro models of BBB are established, including primary bovine or human brain endothelial cells in different cocultures, for example the human brain endothelial cell line HBEC-5i. In vivo, imaging methods, such as CT scans or MRI, can be used together with contrast agents to visualize BBB permeability. Functional imaging, such as PET or SPECT, may also be used.

The VLP according to the invention can be administered via various routes, including oral, dermal, nasal administration or pulmonary routes or parenteral injection (i.v., s.c., i.m.). Particularly preferred are dosage forms which allow a systemic effect of aspartoacylase or galactocerebrosidase, respectively, or the expression vector encoding the respective enzyme or the mRNA encoding the respective enzyme or the combination therof. In a specific embodiment the VLP according to the invention is administered orally or parenterally, in particular intravenously.

In case the application of the VLP according to the invention leads to an unwanted immune reaction, measured for example by the upregulation of inflammatory cytokines and/or surface molecules on immune cells, it may be necessary to additionally apply an immunosuppressant to reduce the activation or efficacy of the immune system.

In a preferred embodiment, the VLP according to the invention, after administration to the subject to be treated, in particular a human, can be detected in the CNS in less than 10 days, preferably in less than 5 days, more preferably in less than 3 days after administration.

In another preferred embodiment, aspartoacylase or galactocerebrosidase, respectively, has a therapeutically effective enzyme activity for at least 10 days, preferably for at least 20 days, more preferably for at least 30 days. The therapeutically effective enzyme activity can be measured by the enzyme activity which leads to a therapeutic effect, i.e. to at least an alleviation or mitigation of a disease symptom.

It is particularly advantageous that the therapeutically effective enzyme activity preferably at the target site is maintained for at least 10 days, preferably for at least 20 days in order to extend the effective period when aspartoacylase or galactocerebrosidase, respectively, exerts its activity. Thereby, the number or frequency of injections of VLP according to the present invention can be limited.

In one embodiment, the aspartoacylase comprises an amino acid sequence which is at least 80 %, preferably at least 90 % identical to the amino acid sequence according to SEQ ID NO: 1 over its entire length, more preferably has the amino acid sequence of SEQ ID NO: 1.

In one embodiment, the aspartoacylase is encoded by a nucleotide sequence which is at least 70 %, preferably at least 80 %, more preferably at least 90 % identical to the nucleotide sequence of SEQ ID NO: 2 over its entire length, most preferably is the nucleotide sequence of SEQ ID NO2.

In one embodiment, the aspartoacylase is encoded by an mRNA sequence which is at least 70 %, preferably at least 80 %, more preferably at least 90 % identical to the mRNA sequence of SEQ ID NO: 9 over its entire length, most preferably comprises the mRNA sequence of SEQ ID NO: 9.

In one embodiment, the galactocerebrosidase comprises an amino acid sequence which is at least 80 %, preferably at least 90 % identical to the amino acid sequence according to SEQ ID NO: 3 over its entire length, more preferably has the amino acid sequence of SEQ ID NO: 3.

In one embodiment, the galactocerebrosidase is encoded by a nucleotide sequence which is at least 70 %, preferably at least 80 %, more preferably at least 90 % identical to the nucleotide sequence of SEQ ID NO: 4 over its entire length, most preferably is the nucleotide sequence of SEQ ID NO: 4.

In one embodiment, the galactocerebrosidase is encoded by an mRNA sequence which is at least 70 %, preferably at least 80 %, more preferably at least 90 % identical to the mRNA sequence of SEQ ID NO: 10 over its entire length, most preferably comprises the mRNA sequence of SEQ ID NO: 10.

The sequences with SEQ ID NOs 1, 2 and 9 pertain to the enzyme human aspartoacylase and the sequences with SEQ ID NOs 3, 4 and 10 to the enzyme human galactocerebrosidase and are shown in the table below:

TABLE 1

Amino acid and nucleotide sequences (DNA and mRNA) of human aspartoacylase and human galactocerebrosidase.

| | | | |
|---|---|---|---|
| Protein / DNA | Sequence | Source | SEQ ID NO: |
| Protein (aspartoacyla se) | MTSCHIAEEHIQKVAIFGGTHGNELTGVFLV KHWLENGAEIQRTGLEVKPFITNPRAVKKC TRYIDCDLNRIFDLENLGKKMSEDLPYEVRR AQEINHLFGPKDSEDSYDIIFDLHNTTSNMG CTLILEDSRNNFLIQMFHYIKTSLAPLPCYVY LIEHPSLKYATTRSIAKYPVGIEVGPQPQGV LRADILDQMRKMIKHALDFI HHFNEGKEFPP CAIEVYKIIEKVDYPRDENGEIAAIIHPNLQD QDWKPLHPGDPMFLTLDGKTIPLGGDCTV | human | 1 |
| | YPVFVNEAAYYEKKEAFAKTTKLTLNAKSIR CCLH | | |
| DNA (aspartoacyla se) | ATGACTTCTTGTCACATTGCTGAAGAACA TATACAAAAGGTTGCTATCTTTGGAGGAA CCCATGGGAATGAGCTAACCGGAGTATTT CTGGTTAAGCATTGGCTAGAGAATGGCG CTGAGATTCAGAGAACAGGGCTGGAGGT AAAACCATTTATTACTAACCCCAGAGCAG TGAAGAAGTGTACCAGATATATTGACTGT GACCTGAATCGCATTTTTGACCTTGAAAA TCTTGGCAAAAAAATGTCAGAAGATTTGC CATATGAAGTGAGAAGGGCTCAAGAAATA AATCATTTATTTGGTCCAAAAGACAGTGA AGATTCCTATGACATTATTTTTGACCTTCA CAACACCACCTCTAACATGGGGTGCACTC TTATTCTTGAGGATTCCAGGAATAACTTTT TAATTCAGATGTTTCATTACATTAAGACTT CTCTGGCTCCACTACCCTGCTACGTTTAT CTGATTGAGCATCCTTCCCTCAAATATGC GACCACTCGTTCCATAGCCAAGTATCCTG TGGGTATAGAAGTTGGTCCTCAGCCTCAA GGGGTTCTGAGAGCTGATATCTTGGATCA AATGAGAAAAATGATTAAACATGCTCTTG ATTTTATACATCATTTCAATGAAGGAAAAG AATTTCCTCCCTGCGCCATTGAGGTCTAT AAAATTATAGAGAAAGTTGATTACCCCCG GGATGAAAATGGAGAAATTGCTGCTATCA TCCATCCTAATCTGCAGGATCAAGACTGG AAACCACTGCATCCTGGGGATCCCATGTT TTTAACTCTTGATGGGAAGACGATCCCAC TGGGCGGAGACTGTACCGTGTACCCCGT GTTTGTGAATGAGGCCGCATATTACGAAA AGAAAGAAGCTTTTGCAAAGACAACTAAA CTAACGCTCAATGCAAAAAGTATTCGCTG CTGTTTACATTAG | human | 2 |
| Protein (galactocereb rosidase) | MAEWLLSASWQRRAKAMTAAAGSAGRAA VPLLLCALLAPGGAYVLDDSDGLGREFDGI GAVSGGGATSRLLVNYPEPYRSQILDYLFK PNFGASLHILKVEIGGDGQTTDGTEPSHMH YALDENYFRGYEWWLMKEAKKRNPNITLIG LPWSFPGWLGKGFDWPYVNLQLTAYYVVT WIVGAKRYHDLDIDYIGIWNERSYNANYIKIL RKMLNYQGLQRVKIIASDNLWESISASMLLD AELFKVVDVIGAHYPGTHSAKDAKLTGKKL | human | 3 |
| | WSSEDFSTLNSDMGAGCWGRILNQNYING YMTSTIAWNLVASYYEQLPYGRCGLMTAQ EPWSGHYWESPVWVSAHTTQFTQPGWY YLKTVGHLEKGGSYVALTDGLGNLTIIIETM SHKHSKCIRPFLPYFNVSQQFATFVLKGSF SEIPELQVWYTKLGKTSERFLFKQLDSLWL LDSDGSFTLSLHEDELFTLTTLTTGRKGSYP LPPKSQPFPSTYKDDFNVDYPFFSEAPNFA DQTGVFEYFTNIEDPGEHHFTLRQVLNQRP ITWAADASNTISIIGDYNWTNLTIKCDVYIET PDTGGVFIAGRVNKGGILIRSARGIFFWIFA NGSYRVTGDLAGWIIYALGRVEVTAKKWYT LTLTIKGHFTSGMLNDKSLWTDIPVNFPKNG WAAIGTHSFEFAQFDNFLVEATR | | |
| DNA (galactocereb rosidase) | AGTCATGTGACCCACACAATGGCTGAGT GGCTACTCTCGGCTTCCTGGCAACGCCG AGCGAAAGCTATGACTGCGGCCGCGGGT TCGGCGGGCCGCGCCGCGGTGCCCTTG CTGCTGTGTGCGCTGCTGGCGCCCGGCG GCGCGTACGTGCTCGACGACTCCGACGG GCTGGGCCGGGAGTTCGACGGCATCGG CGCGGTCAGCGGCGGCGGGGCAACCTC CCGACTTCTAGTAAATTACCCAGAGCCCT ATCGTTCTCAGATATTGGATTATCTCTTTA AGCCGAATTTTGGTGCCTCTTTGCATATTT TAAAAGTGGAAATAGGTGGTGATGGGCA GACAACAGACGGCACTGAGCCCTCCCAC ATGCATTATGCACTAGATGAGAATTATTTC CGAGGATACGAGTGGTGGTTGATGAAAG AAGCTAAGAAGAGGAATCCCAATATTACA CTCATTGGGTTGCCATGGTCATTCCCTGG ATGGCTGGGAAAAGGTTTCGACTGGCCT TATGTCAATCTTCAGCTGACTGCCTATTAT GTCGTGACCTGGATTGTGGGCGCCAAGC GTTACCATGATTTGGACATTGATTATATTG GAATTTGGAATGAGAGGTCATATAATGCC AATTATATTAAGATATTAAGAAAAATGCTG AATTATCAAGGTCTCCAGCGAGTGAAAAT CATAGCAAGTGATAATCTCTGGGAGTCCA TCTCTGCATCCATGCTCCTTGATGCCGAA CTCTTCAAGGTGGTTGATGTTATAGGGGC TCATTATCCTGGAACCCATTCAGCAAAAG ATGCAAAGTTGACTGGGAAGAAGCTTTGG TCTTCTGAAGACTTTAGCACTTTAAATAGT | human | 4 |
| | GACATGGGTGCAGGCTGCTGGGGTCGCA TTTTAAATCAGAATTATATCAATGGCTATA TGACTTCCACAATCGCATGGAATTTAGTG GCTAGTTACTATGAACAGTTGCCTTATGG GAGATGCGGGTTGATGACGGCCCAGGAG CCATGGAGTGGGCACTACGTGGTAGAAT CTCCTGTCTGGGTATCAGCTCATACCACT CAGTTTACTCAACCTGGCTGGTATTACCT GAAGACAGTTGGCCATTTAGAGAAAGGA GGAAGCTACGTAGCTCTGACTGATGGCTT AGGGAACCTCACCATCATCATTGAAACCA TGAGTCATAAACATTCTAAGTGCATACGG CCATTTCTTCCTTATTTCAATGTGTCACAA CAATTTGCCACCTTTGTTCTTAAGGGATCT TTTAGTGAAATACCAGAGCTACAGGTATG GTATACCAAACTTGGAAAAACATCCGAAA GATTTCTTTTTAAGCAGCTGGATTCTCTAT GGCTCCTTGACAGCGATGGCAGTTTCAC ACTGAGCCTGCATGAAGATGAGCTGTTCA CACTCACCACTCTCACCACTGGTCGCAAA GGCAGCTACCCGCTTCCTCCAAAATCCCA GCCCTTCCCAAGTACCTATAAGGATGATT TCAATGTTGATTACCCATTTTTTAGTGAAG CTCCAAACTTTGCTGATCAAACTGGTGTA TTTGAATATTTTACAAATATTGAAGACCCT GGCGAGCATCACTTCACGCTACGCCAAG TTCTCAACCAGAGACCCATTACATGGGCT GCCGATGCATCCAACACAATCAGTATTAT AGGAGACTACAACTGGACCAATCTGACTA TAAAGTGTGATGTATACATAGAGACCCCT GACACAGGAGGTGTGTTCATTGCAGGAA GAGTAAATAAAGGTGGTATTTTGATTAGA AGTGCCAGAGGAATTTTCTTCTGGATTTTT GCAAATGGATCTTACAGGGTTACAGGTGA TTTAGCTGGATGGATTATATATGCTTTAGG ACGTGTTGAAGTTACAGCAAAAAAATGGT ATACACTCACGTTAACTATTAAGGGTCATT TCACCTCTGGCATGCTGAATGACAAGTCT CTGTGGACAGACATCCCTGTGAATTTTCC AAAGAATGGCTGGGCTGCAATTGGAACT CACTCCTTTGAATTTGCACAGTTTGACAA CTTTCTTGTGGAAGCCACACGCTAATACT TAACAGGGCATCATAGAATA | | |
| mRNA (aspartoacyla se) | AGGAAAUAAGAGAGAAAAGAAGAGUAAG AAGAAAUAUAAGAGCCACCAUGACUUCU UGUCACAUUGCUGAAGAACAUAUACAAA AGGUUGCUAUCUUUGGAGGAACCCAUG GGAAUGAGCUAACCGGAGUAUUUCUGG UUAAGCAUUGGCUAGAGAAUGGCGCUG AGAUUCAGAGAACAGGGCUGGAGGUAAA ACCAUUUAUUACUAACCCCAGAGCAGUG AAGAAGUGUACCAGAUAUAUUGACUGUG ACCUGAAUCGCAUUUUUGACCUUGAAAA UCUUGGCAAAAAAAUGUCAGAAGAUUUG CCAUAUGAAGUGAGAAGGGCUCAAGAAA UAAAUCAUUUAUUUGGUCCAAAAGACAG UGAAGAUUCCUAUGACAUUAUUUUUGAC CUUCACAACACCACCUCUAACAUGGGGU GCACUCUUAUUCUUGAGGAUUCCAGGAA UAACUUUUUAAUUCAGAUGUUUCAUUAC AUUAAGACUUCUCUGGCUCCACUACCCU GCUACGUUUAUCUGAUUGAGCAUCCUUC CCUCAAAUAUGCGACCACUCGUUCCAUA GCCAAGUAUCCUGUGGGUAUAGAAGUU GGUCCUCAGCCUCAAGGGGUUCUGAGA GCUGAUAUCUUGGAUCAAAUGAGAAAAA UGAUUAAACAUGCUCUUGAUUUUAUACA UCAUUUCAAUGAAGGAAAAGAAUUUCCU CCCUGCGCCAUUGAGGUCUAUAAAAUUA UAGAGAAAGUUGAUUACCCCCGGGAUGA AAAUGGAGAAAUUGCUGCUAUCAUCCAU CCUAAUCUGCAGGAUCAAGACUGGAAAC CACUGCAUCCUGGGGAUCCCAUGUUUU UAACUCUUGAUGGGAAGACGAUCCCACU GGGCGGAGACUGUACCGUGUACCCCGU GUUUGUGAAUGAGGCCGCAUAUUACGAA AAGAAAGAAGCUUUUGCAAAGACAACUA AACUAACGCUCAAUGCAAAAAGUAUUCG CUGCUGUUUACAUUAGGCGGCCGCUUA AUUAAGCUGCCUUCUGCGGGGCUUGCC UUCUGGCCAUGCCCUUCUUCUCUCCCU UGCACCUGUACCUCUUGGUCUUUGAAUA AAGCCUGAGUAGGAAGUCUAGAGUUUAA ACAUUUAAAUCUGCAGAUCCCAAUGGCG CG | human | 9 |
| mRNA (galactocereb rosidase) | AGGAAAUAAGAGAGAAAAGAAGAGUAAG AAGAAAUAUAAGAGCCACCAUGGCUGAG UGGCUACUCUCGGCUUCCUGGCAACGC CGAGCGAAAGCUAUGACUGCGGCCGCG GGUUCGGCGGGCCGCGCCGCGGUGCCC UUGCUGCUGUGUGCGCUGCUGGCGCCC GGCGGCGCGUACGUGCUCGACGACUCC GACGGGCUGGGCCGGGAGUUCGACGGC AUCGGCGCGGUCAGCGGCGGCGGGGCA ACCUCCCGACUUCUAGUAAAUUACCCAG AGCCCUAUCGUUCUCAGAUAUUGGAUUA UCUCUUUAAGCCGAAUUUUGGUGCCUC UUUGCAUAUUUUAAAAGUGGAAAUAGGU GGUGAUGGGCAGACAACAGACGGCACU GAGCCCUCCCACAUGCAUUAUGCACUAG AUGAGAAUUAUUUCCGAGGAUACGAGUG GUGGUUGAUGAAAGAAGCUAAGAAGAGG AAUCCCAAUAUUACACUCAUUGGGUUGC CAUGGUCAUUCCCUGGAUGGCUGGGAA AAGGUUUCGACUGGCCUUAUGUCAAUCU UCAGCUGACUGCCUAUUAUGUCGUGAC CUGGAUUGUGGGCGCCAAGCGUUACCA UGAUUUGGACAUUGAUUAUAUUGGAAUU UGGAAUGAGAGGUCAUAUAAUGCCAAUU AUAUUAAGAUAUUAAGAAAAAUGCUGAA UUAUCAAGGUCUCCAGCGAGUGAAAAUC AUAGCAAGUGAUAAUCUCUGGGAGUCCA UCUCUGCAUCCAUGCUCCUUGAUGCCG AACUCUUCAAGGUGGUUGAUGUUAUAG GGGCUCAUUAUCCUGGAACCCAUUCAGC AAAAGAUGCAAAGUUGACUGGGAAGAAG CUUUGGUCUUCUGAAGACUUUAGCACUU UAAAUAGUGACAUGGGUGCAGGCUGCU GGGGUCGCAUUUUAAAUCAGAAUUAUAU CAAUGGCUAUAUGACUUCCACAAUCGCA UGGAAUUUAGUGGCUAGUUACUAUGAAC AGUUGCCUUAUGGGAGAUGCGGGUUGA UGACGGCCCAGGAGCCAUGGAGUGGGC ACUACGUGGUAGAAUCUCCUGUCUGGG UAUCAGCUCAUACCACUCAGUUUACUCA ACCUGGCUGGUAUUACCUGAAGACAGUU GGCCAUUUAGAGAAAGGAGGAAGCUACG UAGCUCUGACUGAUGGCUUAGGGAACC UCACCAUCAUCAUUGAAACCAUGAGUCA | human | 10 |
| | UAAACAUUCUAAGUGCAUACGGCCAUUU CUUCCUUAUUUCAAUGUGUCACAACAAU UUGCCACCUUUGUUCUUAAGGGAUCUU UUAGUGAAAUACCAGAGCUACAGGUAUG GUAUACCAAACUUGGAAAAACAUCCGAA AGAUUUCUUUUUAAGCAGCUGGAUUCUC UAUGGCUCCUUGACAGCGAUGGCAGUU UCACACUGAGCCUGCAUGAAGAUGAGCU GUUCACACUCACCACUCUCACCACUGGU CGCAAAGGCAGCUACCCGCUUCCUCCAA AAUCCCAGCCCUUCCCAAGUACCUAUAA GGAUGAUUUCAAUGUUGAUUACCCAUUU UUUAGUGAAGCUCCAAACUUUGCUGAUC AAACUGGUGUAUUUGAAUAUUUUACAAA UAUUGAAGACCCUGGCGAGCAUCACUUC ACGCUACGCCAAGUUCUCAACCAGAGAC CCAUUACAUGGGCUGCCGAUGCAUCCAA CACAAUCAGUAUUAUAGGAGACUACAAC UGGACCAAUCUGACUAUAAAGUGUGAUG UAUACAUAGAGACCCCUGACACAGGAGG UGUGUUCAUUGCAGGAAGAGUAAAUAAA GGUGGUAUUUUGAUUAGAAGUGCCAGA GGAAUUUUCUUCUGGAUUUUUGCAAAUG GAUCUUACAGGGUUACAGGUGAUUUAG CUGGAUGGAUUAUAUAUGCUUUAGGAC GUGUUGAAGUUACAGCAAAAAAAUGGUA UACACUCACGUUAACUAUUAAGGGUCAU UUCACCUCUGGCAUGCUGAAUGACAAGU CUCUGUGGACAGACAUCCCUGUGAAUUU UCCAAAGAAUGGCUGGGCUGCAAUUGG AACUCACUCCUUUGAAUUUGCACAGUUU GACAACUUUCUUGUGGAAGCCACACGCU AAGCGGCCGCUUAAUUAAGCUGCCUUCU GCGGGGCUUGCCUUCUGGCCAUGCCCU UCUUCUCUCCCUUGCACCUGUACCUCUU GGUCUUUGAAUAAAGCCUGAGUAGGAAG UCUAGAGUUUAAACAUUUAAAUCUGCAG AUCCCAAUGGCGCG | | |

Preferably, the expression vector encoding aspartoacylase or galactocerebrosidase, respectively has a size of less than 7 kb, preferably less than 6 kb. The associating of the VLP with the expression vector is more efficient when the size of the expression vector is relatively small, preferably less than 6 kb, more preferably less than 5 kb, most preferably less than 4 kb.

In a preferred embodiment, the expression vector has a promoter selected from the group consisting of CMV and CAG.

The CAG promoter is particularly advantageous because it allows a long-lasting expression. The CAG promoter also inhibits unwanted immune reactions so that the additional application of immunosuppressants may be reduced or even completely avoided.

The CMV promoter is preferred if a stronger and shorter expression is needed.

The need of a long-lasting or short expression may vary depending on the disease, the stage of the disease and the subject to be treated. A long-lasting expression of the enzyme allows a long-lasting effect which is beneficial if there is no or only little residual activity of aspartoacylase or galactocerebrosidase, respectively, in the subject. In case there is residual activity of aspartoacylase or galactocerebrosidase, respectively, a shorter expression may be sufficient. Likewise, the need of a light or strong expression may vary.

Preferably, aspartoacylase or galactocerebrosidase, respectively is expressed, preferably at the target site, for at least 1 h, preferably for at least 5 h, more preferably for at least 12 h, more preferably for at least 1 day, more preferably for at least 3 days, more preferably for at least 1 week, more preferably for at least 1 month, more preferably for at least 3 months, more preferably for at least 6 months, more preferably for at least 9 months, more preferably for at least 1 year, more preferably for at least 1.5 years, more preferably for at least 2 years.

Most preferably, aspartoacylase or galactocerebrosidase, respectively is expressed at the target site for at least 1 month.

In one embodiment therefore, aspartoacylase or galactocerebrosidase, respectively, is detectable at the target site for at least 1 h, preferably for at least 5 h, more preferably for at least 12 h, more preferably for at least 1 day, more preferably for at least 3 days, more preferably for at least 1 week, more preferably for at least 1 month, more preferably for at least 3 months, more preferably for at least 6 months, more preferably for at least 9 months, more preferably for at least 1 year, more preferably for at least 1.5 years, more preferably for at least 2 years.

Most preferably, aspartoacylase or galactocerebrosidase, respectively is detectable at the target site for at least 1 month.

Preferably, the expression in the target cell is transient. A transient expression does not exclude that the expression is long-lasting. In a preferred embodiment, the expression is long-lasting and transient.

In a preferred embodiment, the expression vector is a plasmid.

In a particularly preferred embodiment, the plasmid is free of antibiotic resistance genes. This is advantageous because it allows culturing such plasmids without antibiotics. This is in particular critical for clinical use because the injection of antibiotics may lead to sensitization or to anaphylactic shocks. A “pFAR” plasmid is for example a plasmid without antibiotic resistance genes. A pFAR plasmid allows a long-lasting and stable expression without the need to use antibiotics and might be used for practicing the invention.

In another preferred embodiment, a “pNL” plasmid or a “pSF” plasmid is used.

The expression “pFAR plasmid”, “pNL plasmid” or “pSF plasmid” means that a plasmid with the backbone of a pFAR plasmid, a pNL plasmid or a pSF plasmid is used for cloning the promoter and the enzyme of interest into the backbone.

A pFAR plasmid is for example described in US 8,440,455 B2. A particularly preferred pFAR plasmid is pFAR4, the backbone of which is disclosed as SEQ ID NO: 21 in US 8,440,455 B2. The construction of the “pFAR1” plasmid and the optimized “pFAR4” plasmid is disclosed in columns 17 and 18 of US 8,440,455 B2.

The pFAR plasmid preferably comprises a CMV or a CAG promoter. The pNL plasmid preferably comprises a CMV promoter. The pSF plasmid preferably comprises a CAG promoter.

In a preferred embodiment, the expression vector is pNL-CMV-hASPA or pNL-CMV-hGALC, respectively, thus a pNL backbone with a CMV promoter and encoding the human ASPA or GALC gene, respectively. In another preferred embodiment, the expression vector is pFAR-CAG-hASPA or pFAR-CAG-hGALC. The expression vector pNL-CMV-hASPA or pNL-CMV-GALC, respectively, is particularly preferred.

It has been shown in the present invention that aspartoacylase and galactocerebrosidase, respectively, can be expressed in different cell lines.

In a preferred embodiment, if the cargo is an expression vector encoding aspartoacylase or galactocerebrosidase, respectively, the treatment of Canavan disease and Krabbe disease, respectively, is effected by the transient expression of the respective genes in the target cell, preferably an oligodendrocyte, of the subject to be treated.

In another preferred embodiment, if the cargo is an mRNA encoding aspartoacylase or galactocerebrosidase, respectively, the treatment of Canavan disease and Krabbe disease, respectively, is effected by the transient expression of the respective genes in the target cell, preferably an oligodendrocyte, of the subject to be treated.

Subjects to be treated by the VLP according to the present invention are e.g. patients suffering from Canavan disease or Krabbe disease, respectively, or subjects expected to suffer from said diseases, for example because they encompass a genetic mutation which is known to affect aspartoacylase or galactocerebrosidase, respectively, and thus leads to Canavan disease and Krabbe disease, respectively.

Different mouse models of Canavan disease and Krabbe disease exist which can be used to study the diseases and possible therapeutics:

The ASPA^{nur7} mice do not express the aspartoacylase enzyme due to a nonsense mutation in the ASPA gene. They are characterized by an early-onset spongy degeneration of the myelin in the central nervous system accompanied by an increased NAA level. ASPA^{nur7} mice are much smaller compared to wild type mice and tremors and seizures can be detected (Traka et al., J. Neurosci. 2009; 28: 11537-11549).

The Twitcher mice have a mutation in the GALC gene. Deficient mice are smaller and less active compared to wild type mice with a life span of only about 40 days. A lack of myelin can be detected in the CNS and the PNS. The pathogenesis results from an abormal accumulation of galactosylsphingosine (psychosine) in the nervous system (Suzuki et al., Brain Pathology 1995; 5(3): 249-258).

The VLP of the invention is preferably derived from John Cunningham virus (JCV). The “JC virus” or John Cunningham virus (JCV; NCBI Taxonomy 10632) is a human polyomavirus. JCV is of an icosahedral symmetry, has a diameter of about 45 nm and consists of 72 VP1 pentamers. Small numbers of the structural proteins VP2 and VP3 are also present.

A “virus-like particle” (VLP) in the context of the present invention is defined as a replication-deficient particle with a hull (also termed capsid) composed of viral structural proteins or modified viral structural proteins or proteins derived from viral structural proteins. As explained above, the VLP as such does not comprise genetic material. The VLP according to the invention is preferably derived from a human polyoma virus, preferably JCV, i.e. its hull is preferably composed of viral structural proteins or modified viral structural proteins or proteins derived from viral structural proteins VP1, VP2 and VP3 from JCV. In a preferred embodiment, the VLP according to the invention is composed of VP1 proteins of JC virus.

In a particularly preferred embodiment of the invention the only viral structural protein in the hull of the VLP according to the invention is a VP1 protein. In the most preferred embodiment the hull of the VLP according to the invention consists of VP1 proteins, i.e. the hull does not contain any other protein.

The viral structural proteins, in particular the VP1, assemble into pentameric structures (pentamers). According to the invention, the VLP hull according to the invention preferably is composed of several VP1 proteins, in particular several VP1 pentamers, especially 72 VP1 pentamers.

A “pentamer” in the context of the invention is a structure which is formed when five polypeptides, for example VP1 proteins, assemble. The assembly into a pentamer may be due to the formation of covalent or non-covalent bonds between the polypeptides. The polypeptides typically form a ring-shaped structure, having pentagonal symmetry. In a pentamer, each polypeptide subunit preferably interacts with two adjacent subunits.

A “peptide” according to the present invention may be composed of any number of amino acids of any type, preferably naturally occurring amino acids, which preferably are linked by peptide bonds. In particular, a peptide comprises at least 3 amino acids, preferably at least 5, at least 7, at least 9, at least 12 or at least 15 amino acids. There is no upper limit for the length of a peptide. However, preferably a peptide according to the invention does not exceed a length of 500 amino acids, preferably 400, 300, 250, 200, 150 or 120 amino acids. A peptide exceeding about 10 amino acids may also be termed a “polypeptide”.

The structural proteins of the VLP according to the invention, in particular the VP1, are preferably identical to or derived from the native structural proteins of JCV. “Modified or derived” encompasses the insertion, deletion or substitution of one or more amino acids while retaining the function of VP1 to assemble into a capsid.

In one embodiment, the native (JCV) structural protein can be modified in order to optimize the VLP according to the invention with regard to its production, its cellular targeting profile and specificity or its intracellular targeting profile or specificity. Modification or derivation can comprise a codon optimization of the nucleotide sequence encoding the structural protein, in particular the VP1, to enhance protein translation.

The terms “VP1” or “virus protein 1” according to the present invention refer to a protein which is capable of assembling into a capsid and which is preferably identical to or is derived from the natural (native) VP1 of the JCV.

The term “VP1” according to the invention encompasses a protein which has an amino acid sequence identity with the amino acid sequence according to SEQ ID NO: 5 or 6 of at least 80 %, more preferably at least 85 %, more preferably at least 90 %, more preferably at least 95 %, more preferably at least 97 %, more preferably at least 98 % or at least 99 % over this sequence. In a most preferred embodiment of the invention the VP1 has the amino acid sequence according to SEQ ID NO: 5 or 6.

The term “VP1” according to the invention also encompasses fractions of the native VP1. Preferably, said fractions of VP1 comprise at least amino acids 32 to 316 of the amino acid sequence according to SEQ ID NO: 5 or 6 or a derivative thereof having an identity with the amino acid sequence from amino acid position 32 to 316 of SEQ ID NO: 5 or 6 of at least 80 %, more preferably at least 85 %, more preferably at least 90 %, more preferably at least 95 %, more preferably at least 97 %, more preferably at least 98 % or at least 99 % over this sequence.

In one embodiment, the nucleotide sequence of the VP1 protein is at least 70 %, more preferably at least 80 %, more preferably at least 90 % identical to the nucleotide sequence of SEQ ID NO: 7 over its entire length, preferably is the nucleotide sequence of SEQ ID NO: 7.

In another embodiment of the invention the nucleotide sequence of the VP1 protein is at least 70 %, more preferably at least 80 %, more preferably at least 90 % identical to the nucleotide sequence of SEQ ID NO: 8 over its entire length. In one embodiment, the nucleotide sequence of the VP1 protein is identical to the nucleotide sequence of SEQ ID NO: 8.

The sequences are depicted in Table 2.

TABLE 2

Amino acid and nucleotide sequences of the VP1 protein.

| | | | |
|---|---|---|---|
| Protein / DNA | Sequence | Source | SEQ ID NO: |
| Protein | MAPTKRKGEPKDPVQVPKLLIRGGVEVLE VKTGVDSITEVECFLTPEMGDPDEHLRGF SKSISISDTFESDSPNRDMLPCYSVARIPL PNLNEDLTCGNILMWEAVTLKTEVIGVTSL MNVHSNGQATHDNGAGKPVQGTSFHFFS VGGEALELQGVVFNYRTKYPDGTIFPKNA TVQSQVMNTEHKAYLDKNKAYPVECWVP DPTRNENTRYFGTLTGGENVPPVLHITNT ATTVLLDEFGVGPLCKGDNLYLSAVDVCG MFTNRSGSQQWRGLSRYFKVQLRKRRV KNPYPISFLLTDLINRRTPRVDGQPMYGM DAQVEEVRVFEGTEELPGDPDMMRYVDK YGQLQTKML | derived from JCV | 5 |
| Protein | MAPTKRKGERKDPVQVPKLLIRGGVEVLE VKTGVDSITEVECFLTPEMGDPDEHLRGF SKSISISDTFESDSPSKDMLPCYSVARIPLP NLNEDLTCGNILMWEAVTLKTEVIGVTSLM NVHSNGQAAHDNGAGKPVQGTSFHFFSV GGEALELQGVVFNYRTKYPDGTI FPKNAT VQSQVMNTEHKAYLDKNKAYPVECWVPD PTRNENTRYFGTLTGGENVPPVLHITNTA TTVLLDEFGVGPLCKGDNLYLSAVDVCGM FTNRSGSQQWRGLSRYFKVQLRKRRVKN PYPISFLLTDLINRRTPRVDGQPMYGMDA QVEEVRVFEGTEELPGDPDMMRYVDRYG QLQTKML | wildtype JCV (AFH571 94.1) | 6 |
| DNA | ATGGCTCCCACCAAGCGCAAGGGCGAG CCCAAGGACCCCGTGCAAGTGCCCAAG CTGCTGATCCGTGGTGGTGTCGAGGTG CTGGAAGTCAAGACCGGCGTGGACTCC ATTACCGAGGTGGAGTGCTTCCTCACCC CCGAGATGGGTGACCCTGACGAGCACC TGAGGGGCTTCTCCAAGTCCATCTCCAT CTCCGACACCTTCGAGTCCGACTCCCC CAACCGTGACATGCTGCCCTGCTACTCC GTGGCTCGTATCCCCCTGCCCAACCTG AACGAGGACCTGACTTGCGGCAACATC CTGATGTGGGAGGCTGTGACCCTCAAG ACCGAGGTCATCGGCGTGACTTCCCTG ATGAACGTGCACTCCAACGGCCAGGCT ACCCACGACAACGGTGCTGGCAAGCCC GTGCAGGGAACCTCCTTCCACTTCTTCT CCGTGGGTGGCGAGGCTCTGGAACTCC AGGGCGTGGTGTTCAACTACCGTACCAA GTACCCCGACGGCACCATCTTCCCCAA GAACGCTACTGTGCAGTCCCAAGTGATG AACACCGAGCACAAGGCTTACCTGGAC AAGAACAAGGCCTACCCCGTGGAGTGC TGGGTGCCCGACCCCACCCGTAACGAG AACACCCGTTACTTCGGCACCCTGACCG GTGGAGAGAACGTGCCCCCCGTGCTGC ACATCACCAACACCGCTACCACCGTGCT GCTGGACGAGTTCGGTGTCGGTCCCCT GTGCAAGGGCGACAACCTGTACCTGTC CGCTGTGGACGTGTGCGGCATGTTCAC CAACCGTTCCGGTTCCCAGCAGTGGCG TGGCCTGTCCCGCTACTTCAAGGTGCA GCTGCGCAAGCGTCGTGTGAAGAACCC CTACCCTATCTCCTTCCTGCTGACCGAC CTGATCAACCGTCGTACCCCTCGTGTGG ACGGCCAGCCCATGTACGGCATGGACG CTCAGGTGGAAGAGGTCCGCGTGTTCG AGGGCACCGAGGAATTGCCCGGCGACC CCGACATGATGCGTTACGTGGACAAGTA CGGCCAGCTCCAGACCAAGATGCTGTA A | derived from JCV | 7 |
| DNA | ATGGCCCCAACAAAAAGAAAAGGAGAAA GGAAGGACCCCGTGCAAGTTCCAAAAC TTCTTATAAGAGGAGGAGTAGAAGTTCT AGAAGTTAAAACTGGGGTTGACTCAATT | wildtype JCV (J02226) | 8 |
| | ACAGAGGTAGAATGCTTTTTAACTCCAG AAATGGGTGACCCAGATGAGCATCTTAG GGGTTTTAGTAAGTCAATATCTATATCAG ATACATTTGAAAGTGACTCCCCAAATAG GGACATGCTTCCTTGTTACAGTGTGGCC AGAATTCCACTACCCAATCTAAATGAGG ATCTAACCTGTGGAAATATACTCATGTG GGAGGCTGTGACCTTAAAAACTGAGGTT ATAGGGGTGACAAGTTTGATGAATGTGC ACTCTAATGGGCAAGCAACTCATGACAA TGGTGCAGGGAAGCCAGTGCAGGGCAC CAGCTTTCATTTTTTTTCTGTTGGGGGG GAGGCTTTAGAATTACAGGGGGTGCTTT TTAATTACAGAACAAAGTACCCAGATGG AACAATTTTTCCAAAGAATGCCACAGTG CAATCTCAAGTCATGAACACAGAGCACA AGGCGTACCTAGATAAGAACAAAGCATA TCCTGTTGAATGTTGGGTTCCTGATCCC ACCAGAAATGAAAACACAAGATATTTTG GGACACTAACAGGAGGAGAAAATGTTCC TCCAGTTCTTCATATAACAAACACTGCCA CAACAGTGTTGCTTGATGAATTTGGTGT TGGGCCACTTTGCAAAGGTGACAACTTA TACTTGTCAGCTGTTGATGTCTGTGGCA TGTTTACAAACAGGTCTGGTTCCCAGCA GTGGAGAGGACTCTCCAGATATTTTAAG GTGCAGCTAAGGAAAAGGAGGGTTAAA AACCCCTACCCAATTTCTTTCCTTCTTAC TGATTTAATTAACAGAAGGACTCCTAGA GTTGATGGGCAGCCTATGTATGGCATG GATGCTCAAGTAGAGGAGGTTAGAGTTT TTGAGGGAACAGAGGAGCTTCCAGGGG ACCCAGACATGATGAGATACGTTGACAA ATATGGACAGTTGCAGACAAAAATGCTG TAA | | |

In a preferred embodiment of the invention, the VP1 has an amino acid sequence which is at least 90 % identical to the amino acid sequence according to SEQ ID NO: 5 or 6 over its entire length.

In a preferred embodiment of the invention, the nucleotide sequence of the VP1 protein is at least 80 % identical to the nucleotide sequence of SEQ ID NO: 7 or 8 over its entire length.

The structural proteins, preferably VP1, can be expressed in, for example, E. coli or in insect cells. According to a preferred embodiment of the invention, the structural proteins, preferably VP1, are expressed in insect cells. This is advantageous because the expression in insect cells leads to fewer modifications, such as post-translational modifications, compared with the wildtype protein, for example from JCV, than the expression in E. coli.

The VLP according to the invention can furthermore comprise in the capsid one or several additional heterologous proteins, i.e. proteins which are not identical to or derived from the source of the VP1, e.g. JCV. For example, a heterologous protein can be anchored in the capsid, i.e. at least part of this protein being preferably accessible from the outside. In principle any protein is suitable as such a heterologous protein as long as the heterologous protein can be incorporated into the capsid and does not interfere substantially with the assembly of the VLP according to the invention.

The VLP according to the invention is associated with a cargo, i.e. with aspartoacylase or galactocerebrosidase, respectively, or an expression vector encoding the respective enzyme or an mRNA encoding the respective enzyme or a combination thereof. This means that the cargo is reversibly bound to the VLP. This can e.g. either be due to a physicochemical interaction with or attachment to any part of the capsid or by incorporation of the cargo into the capsid. The incorporation can be complete or incomplete. In a particular preferred embodiment of the invention the major part of the total amount of the cargo is fully incorporated into the capsid. Most preferred is that the cargo is fully encapsulated in the capsid of the VLP according to the invention.

In the context of the invention, the expression that the “VLP comprises the cargo” is used synonymously with the expression that the VLP is “associated with the cargo”. The association of the VLP and the cargo can be the result of “loading” or “packing” the VLP with the cargo.

“Loading” means any process which leads to the association of VLP and cargo, e.g. by osmotic shock or by assembly of VP1 or VP1 pentamers into VLP together with the cargo. “Loaded VLP” are the VLP resulting from this process. The term “packing” relates to the process of loading the VLP via assembly of VP1 or VP1 pentamers into VLP together with the cargo. The VLP resulting therefrom are termed “packed” VLP.

The term “cargo” is used, in the context of the present invention, for an enzyme, in particular aspartoacylase and galactocerebrosidase, or an expression vector encoding such an enzyme or an mRNA encoding such an enzyme or a combination thereof.

In keeping with the above, a VLP packed with an enzyme or an expression vector encoding the enzyme or an mRNA encoding the enzyme or a combination thereof, in particular a VLP packed with aspartoacylase or galactocerebrosidase or a VLP packed with an expression vector encoding aspartoacylase or galactocerebrosidase or a VLP packed with an mRNA encoding aspartoacylase or galactocerebrosidase or a combination thereof might also be referred to as “encapsulated aspartoacylase (ASPA)” and “encapsulated galactocerebrosidase (GALC)”, respectively.

In a special embodiment of the invention, the VLP according to the invention is part of a pharmaceutical composition for use in a method for the treatment of Canavan disease or Krabbe disease, respectively in a subject, wherein the pharmaceutical composition further comprises a pharmaceutically acceptable carrier, and/or excipient.

In a preferred embodiment, the pharmaceutical composition comprises VLP according to the invention, a salt and a buffer and has a pH of between 7.0 and 8.0, preferably around 7.5. The pharmaceutical composition preferably comprises:
a. 120 mM to 170 mM NaCl, preferably 150 mM NaCl,
b. 1 to 5 mM CaCl₂, preferably 2 mM CaCl₂, and
c. 5 to 30 mM Tris-HCl, preferably 10 to 25 mM Tris-HCl, more preferably 10 mM Tris-HCl.

This pharmaceutical composition allows handling the VLP according to the invention under physiological conditions. Under these conditions the VLP according to the invention essentially remain intact, preferably they essentially maintain their capsid structure. If loaded with cargo, such as aspartoacylase or galactocerebrosidase, respectively, or an expression vector encoding the respective enzyme, or an mRNA encoding the respective enzyme, or a combination thereof, the VLP according to the invention essentially remain associated with the cargo. The pharmaceutical composition is especially suitable as a pharmaceutical composition for the intravenous administration of the VLP according to the invention to a subject, in particular to a human.

In a further aspect, the invention relates to an expression vector having a coding region encoding aspartoacylase or galactocerebrosidase, respectively, a promoter selected from the group comprising CAG and CMV, and having a size of less than 7 kb, preferably less than 6 kb, more preferably less than 5 kb, most preferably less than 4 kb.

In a preferred embodiment, the aspartoacylase or galactocerebrosidase, respectively, encoded by the expression vector, i. e. the coding region, comprises a nucleotide sequence which is at least 70 %, more preferably at least 80 %, more preferably at least 90 % identical to the nucleotide sequence of SEQ ID NO: 2 or SEQ ID NO: 4, respectively, over its entire length, preferably is the nucleotide sequence of SEQ ID NO: 2 or SEQ ID NO: 4, respectively.

In case the VLP according to the invention are not immediately administered after manufacture, they can be stored, preferably in liquid nitrogen.

The VLP according to the invention can be characterized according to standard methods, for example by a Bradford assay, HA, DLS, nDSF, HPLC-SEC, AF4, TEM.

The invention also relates to a method of treating Canavan disease or Krabbe disease, with the VLP according to the invention. The method of treating preferably comprises the step of administering the VLP to a subject in need thereof.

The invention also relates to the use of the VLP according to the invention for the manufacture of a medicament for the treatment of Canavan disease or Krabbe disease, respectively. The method of treatment preferably does not comprise a step of increasing the permeability of the BBB of the subject to be treated. The VLP of the invention, preferably, is administered to a patient who has not received any chemical or physical treatment for impairing or disrupting the BBB.

The invention also relates to VLP according to the invention which are used to deliver aspartoacylase or galactocerebrosidase, respectively, or an expression vector encoding the respective enzyme or an mRNA encoding the respective enzyme or a combination thereof across the BBB to the CNS, in particular to cells of the CNS, for example astrocytes, oligodendrocytes, neurons and microglia.

Importantly, the crossing of the BBB by VLP according to the invention enables the VLP to exhibit its function of targeting specific cell populations within the brain, i.e. deliver aspartoacylase or galactocerebrosidase, respectively, or an expression vector encoding the respective enzyme or an mRNA encoding the respective enzyme or a combination thereof to target cells, preferably astrocytes, oligodendrocytes, neurons and microglia. In the context of the invention said VLP comprises a delivery to and/or into the target cells.

In a further embodiment, the invention relates to a method of associating a VLP with an expression vector encoding aspartoacylase or galactocerebrosidase, respectively, wherein the method comprises the following steps:
providing VLP, in particular derived from JCV
exposing the VLP to conditions dissociating the VLP into pentamers
exposing the pentamers to the expression vector in a ratio of VLP to expression vector of 1 to 0.5 to 1 to 0.1, preferably in a ratio of 1 to 0.2, and to conditions inducing the pentamers to assemble into a VLP associated with the expression vector
optionally purifying the VLP
performing a dialysis step.

In another preferred embodiment, a method of associating a VLP with aspartoacylase or galactocerebrosidase, respectively, or an expression vector encoding the respective enzyme, or an mRNA encoding the respective enzyme or a combination thereof is provided, wherein the method comprises the following steps:
a) providing a composition comprising VP1 proteins,
b) exposing the VP1 proteins of the composition of a) to conditions inducing the VP1 to assemble into VLP,
c) exposing the VLP of the composition of b) to conditions disassembling the VLP into pentamers,
d) exposing the pentamers of the composition of c) to conditions inducing the pentamers to reassemble into VLP
e) exposing the VLP of the composition of d) to conditions disassembling the VLP into pentamers,
f) exposing the pentamers of the composition of e) to aspartoacylase or galactocerebrosidase, respectively, or the expression vector or the mRNA or the combination thereof to conditions inducing the pentamers to assemble into a VLP associated with the respective enzyme or the expression vector or the mRNA or the combination thereof.

The ratio of VLP to expression vector (i.e. the packaging ratio) can be varied depending on the specific need. For example, the efficiency of VLP formation or gene expression may be dependent on the ratio. Preferably, a ratio of VLP to expression vector of 1 to 0.5 to 1 to 0.1, more preferably of 1 to 0.2 is used. The person skilled in the art will tailor the ratio to the specific expression vector, preferably plasmid, and desired usage. A packaging ratio of 1 to 0.2 is preferred.

The ratio of VLP to mRNA (i.e. the packaging ratio) can be 1 to 0.2.

In the context of the present invention, and for the ease of explanation, the VLP resulting from step b) may also be termed “pVLP” (primary VLP). The VLP resulting from step d) may also be termed “rVLP” (reassembled VLP). The VLP as the result of step f) may also be termed “cVLP” (cargoVLP). According to the present invention, cargo is an enzyme, in particular aspartoacylase or galactocerbrosidase, preferably human aspartoacylase or human galactocerebrosidase, or an expression vector encoding such an enzyme, preferably human aspartoacylase or human galactocerebrosidase, or an mRNA encoding such an enzyme, preferably human aspartoacylase or human galactocerebrosidase, or a combination thereof.

In another aspect of the invention, the invention relates to VLP obtainable by the method above.

In another aspect of the invention, the invention relates to a composition comprising VLP according to the invention derived from JCV characterized by one or more of the following parameters:
a. a polydispersity index (PDI) of less than 0.3, preferably less than 0.2, preferably less than 0.1, more preferably in a range between 0.01 and 0.09,
b. at least 70 % of VLP with an average diameter from 20 nm to 70 nm, preferably of 30 nm to 70 nm, more preferably of 35 nm to 65 nm, more preferably of 40 to 60 nm,
c. a VLP content within the composition of at least 80 % (v/v), preferably at least 85 % (v/v), preferably at least 90 % (v/v), preferably at least 95 % (v/v).

In another aspect, the invention relates to a drug delivery system obtainable by the method according to the invention. The drug delivery system can be used in a method of therapy and/or diagnosis, preferably for the treatment of neurological disorders, namely a leukodystrophy, in particular Canavan disease or Krabbe disease. Hence the invention also relates to a method of treatment of a disorder, in particular a CNS disease, with the drug delivery system according to the invention. The method of treatment preferably comprises the step of administering the drug delivery system to a subject in need thereof.

The drug delivery system preferably has an improved efficacy. The VLP according to the invention can cross the BBB without a prior increase of the permeability of the BBB. Hence, the drug delivery system of the invention can be used in a method of treatment of a CNS disease, wherein the method does not comprise a step of increasing the permeability of the BBB of the subject to be treated. The drug delivery system of the invention, preferably, is administered to a patient who has not received any chemical or physical treatment for impairing or disrupting the BBB.

In yet a further aspect of the invention a composition comprising VLP is provided, which has least one, preferably all, of the following characteristics (“target parameters”):

TABLE 3

Preferred characteristics of the VLP and the VLP-containing composition according to the invention. AUC = area under the curve

| | | |
|---|---|---|
| Methods for evaluation | Measurements | Target Parameters |
| Transmission Electron Microscopy (TEM) | VLP per grid mesh | >50 particles |
| | Shape of particles | round and enclosing |
| | Diameter of particles | 40-50 nm |
| | Aggregates | Not visible |
| SDS PAGE and Western Blot (WB) | VP1 band | 40 kDa band is observed |
| | VP1 degradation | Minor or no degradation |
| Dynamic Light Scattering (DLS) | PDI | <0.2 |
| | Single peak (volume based distribution) | Yes |
| | Z-average diameter | 40-50 nm |
| | Other peaks (volume based distribution) | Not detectable |
| Bioanalyzer | VP1 purity (40 kDa) | >90% |
| Thermal Shift Assay (TSA) | Major melting peak (in | >57° C. |
| | Tris-HCl-Buffer) Minor melting peaks | Not detectable |
| Nano Differential Scanning Fluorometry (nDSF) | Major inflection peak | >69° C. |
| | Minor inflection peaks | Not detectable |
| Size Exclusion HPLC (SE-HPLC) | Aggregates | <5% of total AUC |
| | Other impurities | <5% of total AUC |
| Field Flow Fractionation (FFF-MALS) | Concentration of | >1.0 x 10¹¹ VLP/mL |
| | particles | 40-50 nm |
| | Size of particles | <5% of total AUC |
| | Aggregates Other impurities | <5% of total AUC |

In one embodiment, the VLP according to the invention show a major inflection peak in nDSF analyses of >67° C.

In one embodiment, AF4 analyses of the VLP according to the invention reveal less than 20 % aggregates and less than 15 % tinies. At least 70 % are VLP of 40 to 50 nm in size.

The drug delivery system of the invention can be administered via various routes, including oral, dermal, nasal or pulmonary routes or injection. Particularly preferred are dosage forms which allow a systemic effect of the pharmaceutical product. In a specific embodiment the drug delivery system of the invention is administered orally or parenterally, in particular intravenously.

In the context of the invention, the term “drug delivery system” refers to a composition for administering a pharmaceutical product to a subject in the need thereof, in particular to a human or animal. A drug delivery system, advantageously, enables the delivery of the pharmaceutical product contained therein or attached thereto to a site of interest, preferably in a human or animal. Preferably the delivery is selective for the target, i.e. more of the pharmaceutical product is delivered to the target than to other sites of the body or organ.

“A drug delivery system for the CNS” means that the drug delivery system selectively targets the CNS.

According to the invention the expression “exposing” something (e.g. the VP1, pentamers, the VLP) to conditions for affecting something (e.g. inducing the assembly) refers to bringing the material under consideration (e.g. the VP1, the pentamers, the VLP) to conditions which can cause this certain effect (e.g. inducing the assembly). Such exposure may be performed by changing the conditions for the material, e.g. by bringing the material into contact with a different buffer, salt or pH etc. This is possible either by adding something to the composition comprising the material or vice versa or by separating the material from the composition and then adding the material to a different composition.

A change of conditions can also be achieved by varying temperature, radiation etc. Naturally, such means for a change of conditions can be combined and/or repeated. Other suitable conditions that induce the desired effect, such as the assembly of the VP1 or pentamers to VLP and/or inducing aggregation of the VLP, are also well known to the skilled person. The same applies to a suitable duration of the exposure to the respective conditions; this can be found out by ordinary means of the skilled person.

The expression “exposing something to conditions for affecting something” does not require the effect to be completed, i.e. not all of the material has to accomplish the effect under consideration. For example, “conditions inducing the pentamers to aggregate” essentially means that the conditions are suitable to induce aggregation. It does not require that indeed all pentamers aggregate.

As used herein, the term “assembly” or “assemble into VLP” means that the structures under consideration (either the VP1 proteins or the pentamers) associate and establish the capsid of the VLP. If the VP1 are used as the starting material the assembly into VLP may include the prior formation of pentamers, meaning that the VP1 proteins may first form pentamers and then form VLP or they may directly assemble into a VLP. The assembly to VLP is reversible.

The term “disassembly”, in turn, refers to a process, when the capsid of the VLP at least partially disintegrates into pentameric structures and/or the structural proteins. The disassembly may be induced by increasing the temperature, by adding proteases and/or by decreasing intermolecular interactions used to form the VLP such as intermolecular disulfide bridges (e.g. by adding reducing agents or adding chelating agents). Such conditions may also include stepwise exposure to a condition. For instance, the composition may be contacted with a reducing agent before the temperature is increased.

Methods of inducing the VP1 and/or pentamers to assemble into VLP are generally known to the skilled person (Goldmann et al. (J. Virol. 1999; 73(5): 4465-69); DE 195 43 553 A1). The same applies to the disassembly of VLP into pentamers. The skilled person, hence, is aware of methods for the control of the assembly and disassembly of VLP.

In one embodiment of the invention the concentration of Ca²⁺ ions in the composition containing the VP1 or pentamers is used for the control of the assembly/disassembly of the VLP. For example, in order to induce the assembly, the concentration of free Ca²⁺ ions can be increased. If the disassembly is desired, the concentration of free Ca²⁺ ions can be lowered by adding a chelating agent to the composition.

A further option for inducing the assembly is to increase the concentration of VP1 pentamers in order to facilitate the assembly into VLP, for example by reducing the solvent in the composition comprising the pentamers. This might require an adaption of the concentration of alkaline earth metals, such as Ca²⁺ or Mg²⁺.

According to a preferred embodiment of the invention, disassembly can be induced by exposing the VLP to conditions under which intermolecular disulfide bridges are reduced, for example by exposing the VLP to reducing conditions. In a preferred embodiment this step is accomplished in the additional presence of a chelating agent. More preferred, the disassembly is induced by exposing the VLP to reducing conditions in the presence of a chelating agent and optionally at an increased temperature.

In a particular embodiment of the invention, the VLP are exposed to a composition comprising DTT and EDTA and/or EGTA, preferably at a temperature of 15° C. to 30° C., preferably 20° C. to 25° C., most preferably at a temperature of about 23° C.

According to a preferred embodiment of the invention, the pentamers of the composition of c) are exposed to conditions inducing the aggregation thereof. This step is most suitable if performed before step d). In a preferred embodiment, at least 20 % of the material (e.g. the pentamers) aggregates, preferably at least 30 %, more preferably at least 40 %.

It has surprisingly been found that this step can lead to a more homogeneous size distribution of the VLP. This allows for a better quality management and standardization, which is of utmost importance if the VLP are used in a drug delivery system. Accordingly, this additional procedure preferably is part of the quality control requirements of a drug delivery system.

The term “aggregate” means any particulate structure. “Aggregation” means a process leading to aggregates. This process is reversible.

The aggregation of the pentamers or VLP can be determined by an increased average particle size of the VLP in the composition compared to the control. The larger particle size may be determined by standard methods, such as dynamic light scattering (DLS).

According to a particular embodiment of the invention, the aggregation of the pentamers or VP1 can be induced by one or more agents which are known in the art to facilitate precipitation of proteins (precipitation agent). Most preferred, according to the invention, thus is the use of a precipitation agent.

A “precipitation agent” refers to an agent that promotes aggregation of VP1 or pentamers. The concept of precipitation agents generally is known to the person skilled on the art. Precipitation agents are typically used to facilitate the concentration and purification of proteins. Precipitation can be the result of altering the solvation potential of the solvent, more specifically, by lowering the solubility of the protein. The solubility may also be decreased by adjusting the pH of the composition to the isoelectric point of a protein. Furthermore, lowering the temperature of the composition can also decrease the solubility of a protein.

Possible precipitation agents are e.g. polyethylene glycol (PEG), or alcohol, for example ethanol, and salts. The latter are known to the skilled person as “agents for salting out”.

Preferably, according to the invention, the precipitation agent is a salt. Most preferred are salts comprising ions known as the “Hofmeister series”. The Hofmeister series describes the ordering of ions with respect to their hydrophobic effect on a specific protein in terms of their ability to affect the solubility of said protein in solution. Ions exerting a hydrophobic effect on a protein are especially preferred. Herein, such ions are referred to as kosmotropic ions.

A precipitation agent comprising at least one kosmotropic anion or cation is preferred. Preferred anions are selected from the group consisting of citrate (C₆H₅O₇³⁻), phosphate (PO₄³⁻), sulfate (SO₄²⁻ ), hydrogen phosphate (HPO₄²⁻), dihydrogen phosphate (H₂PO₄-), iodate (IO₃⁻ ), hydroxide (OH⁻), fluoride (F⁻), bromate (BrO₃⁻) or acetate (CH₃COO⁻) or combinations thereof, the more preferred anions are citrate, phosphate or sulfate, the most preferred anion is sulfate.

Preferred cations are ammonium or quaternary ammonium compounds (NR₄⁺ with R being an alkyl or an aryl group), such as tetramethylammonium ((CH₃)₄N⁺) or dimethylammonium ((CH₃)₂N₂⁺). Further preferred cations are selected from the list comprising potassium (K⁺), caesium (Cs⁺), rubidium (Rb⁺) or lithium (Li⁺) or combinations thereof, particularly preferred are quaternary ammonium compound or ammonium, most preferred is ammonium.

Therefore, the salt preferably comprises an anion and a cation selected from the group consisting of citrate (C₆H₅O₇³⁻), phosphate (PO₄³⁻), sulfate (SO₄²⁻), hydrogen phosphate (HPO₄²⁻), dihydrogen phosphate (H₂PO₄-), iodate (IO₃⁻ ), hydroxide (OH⁻), fluoride (F⁻), bromate (BrO₃⁻ ) or acetate (CH₃COO⁻), quaternary ammonium compounds (NR₄⁺) with R being an alkyl or an aryl group, preferably tetramethylammonium ((CH₃)₄N⁺) or dimethylammonium ((CH₃)₂N₂⁺), ammonium (NH₄⁺), potassium (K⁺), caesium (Cs⁺), rubidium (Rb⁺) or lithium (Li⁺), preferably comprising SO₄²⁻ and/or NH₄⁺ or combinations thereof.

According to a preferred embodiment the salt is selected from the group consisting of (NH₄)₂SO₄, K₂SO₄, Na₂SO₄, (NH₄)₂HPO₄, K₂HPO₄ and Na₂HPO₄. The most preferred salt is ammonium sulfate ((NH₄)₂SO₄).

According to the invention, the aggregation of the pentamers can be induced by any means for bringing the pentamers into contact with the precipitation agent, for example by adding the precipitation agent to the composition comprising the pentamers or vice versa, namely adding the composition comprising the pentamers to a precipitation agent. Also other means, such as a dialysis so that the precipitation agent reaches the pentamers by diffusion, is possible.

According to one preferred embodiment of the present invention, the aggregation of the pentamers is induced by a dialysis against a composition comprising the precipitation agent, for example a composition comprising ammonium sulfate.

According to a particularly preferred embodiment of the invention, the composition containing the pentamers for aggregation, has an ammonium sulfate concentration between 0.3 to 5 M, preferably up to 4 M, even more preferred is a concentration between 1.8 and 2.2 M. Most preferred is around 2 M.

The step of inducing aggregation of the pentamers preferably has a duration of at least 1 hour, more preferred of at least 5 hours, even more preferred of at least 12 hours, most preferred of at least 16 hours. It is preferred that the step has a duration is less than 24 hours. In a preferred embodiment the duration of this step is between 14 and 19 hours. In a most preferred embodiment the duration of this step is between 16 and 18 hours. During this time the pentamers are exposed to conditions for inducing aggregation, in particular they are in contact with ammonium sulfate.

After the step of inducing the aggregation of the pentamers, it is advantageous to include into the process of the invention a step of separating the pentamers from the conditions which had been used for inducing the aggregation. The methods applicable for such a step are not particularly limited; any method known to the skilled person, which allows for the separation of the pentamers from the aggregation inducing conditions is applicable.

In a preferred embodiment of the invention the pentamers are separated from the conditions inducing their aggregation by dialysis. Dialysis can be used if the aggregation of the pentamers is induced by using a precipitation agent. The principle of dialysis can also favorably be applied in order to bring the pentamers into contact with a precipitation agent. Most preferred is, if the method according to the invention includes at least two steps of dialysis: a first dialysis of the composition of step c) against a composition comprising a precipitation agent, and a second dialysis after the induction of aggregation against a composition which is essentially free of the precipitation agent.

The dialysis for separating the pentamers from the precipitation agent preferably is against a composition which is at least similar to physiological conditions. Such a composition preferably comprises a salt and has a pH of 6 to 8.5, preferably of 6.5 to 8.5, more preferably of 7 to 8, most preferably of 7.2 to 7.5, in particular 7.5. The osmolarity of the composition is preferably between 280 and 310 mosmol/l, most preferably 308 mosmol/l. The composition may for example have a saline (sodium chloride) concentration of 0.8 to 0.92 % (w/v), preferably of 0.9 % (w/v).

Separating the pentamers from the conditions which had been used for inducing the aggregation is preferably performed for at least 1 hour, more preferably for at least 5 hours, 12 hours, more preferably for at least 18 hours, more preferably for about 24 hours or longer. Longer time periods are also possible inter alia depending on the concentration of the pentamers which had been induced to aggregate, the composition comprising the pentamers and the nature and concentration of the precipitation agent. In a preferred embodiment, the composition comprising the aggregated pentamers is dialyzed against a composition similar to physiological conditions for about 24 hours.

The composition preferably further contains a buffer. Suitable buffering systems are known to the skilled person. In a preferred embodiment of the invention the composition includes a TRIS buffer, HEPES buffer, a phosphate buffer or a bicarbonate buffer system. Most preferred is a TRIS buffer.

In a most preferred embodiment the composition comprises 10 mM Tris-HCl and 150 mM NaCl and has a pH of 7.5.

In order to facilitate assembly of the pentamers into VLP, the composition may further comprise divalent ions, such as Ca²⁺ Mg²⁺, Ba²⁺, Cu²⁺, Fe²⁺, Zn²⁺ or combinations thereof. Most preferred is Ca²⁺, for example CaCl₂. In a preferred embodiment, the composition comprises 1 to 3 mM CaCl₂, preferably 2 mM CaCl₂.

In a very preferred embodiment of the invention, the composition which is at least similar to physiological conditions comprises 10 mM Tris-HCl, 150 mM NaCl and 2 mM CaCl₂ and has a pH of 7.5.

In yet another aspect of the invention it has surprisingly been found that the storage of VLP (rVLP) is advantageous compared with the storage of pentamers. If pentamers are stored and subsequently thawed and reassembled, predominantly “tiny” particles form as well as aggregates. These VLP are not suitable for the production of a drug delivery system. VLP, however, that have been dissociated and reassembled after storage form a particularly homogeneous population of adequately sized VLP according to the invention. Thus, in one embodiment of the invention a composition is provided with particles having an average diameter from 20 nm to 70 nm, preferably of 30 nm to 70 nm, more preferably of 35 nm to 65 nm, more preferably of 40 to 60 nm. A homogeneous size distribution is important in order to fulfil quality control requirements.

In a preferred embodiment, the method according to the invention comprises a step of storing the VLP from the composition of step d). Storing the VLP at a temperature of about -80° C. to about 4° C. is possible for a duration of at least 10 h, 15 h, 20 h, preferably for at least 24 h. A storage of even more than 3 days is possible.

In a preferred embodiment the VLP are stored at a temperature below 0° C. (freezing). Freezing may be performed using different cooling rates. For example “slow” freezing may occur by applying a cooling rate of about -1° C. per minute, while fast freezing may be performed by contacting the sample, i.e. the container which comprises the composition, with liquid nitrogen or by placing the sample in a freezer at -80° C.

In a preferred embodiment, storing takes place in a composition comprising a cryoadditive, preferably selected from the group consisting of polyols, sugars, inorganic salts, organic salts, amino acids, polymers, extremolytes or derivatives or combinations thereof.

In a preferred embodiment, the inorganic salt comprises a sulfate anion. Preferred salts comprising a sulfate anion are potassium sulfate, sodium sulfate, sodium thiosulfate, magnesium sulfate and ammonium sulfate. Preferably, the inorganic salt is ammonium sulfate.

The amino acid preferably is glycine, glutamine, proline or alanine. A preferred amino acid derivative is betain. Further possible cryoadditives are glycerol, sucrose, DMSO, ectoin or hydroxyectoin.

It has been found that the addition of cryoadditives, in particular the addition of an inorganic salt (such as a salt comprising a sulfate anion, in particular ammonium sulfate) and/or an amino acid derivative (such as betain), is advantageous with respect to the stability and the functionality or efficacy of the VLP. As stated supra, an enhanced stability and/or functionality or efficacy is particularly desired when using VLP as a drug delivery system. It was surprising that the addition of cryoadditives to the composition of step d) has an impact on the packed VLP of step f) in terms of stability and functionality or efficacy.

A cryoadditive serves the purpose of protecting biological tissue from freezing damage (i.e. due to ice formation). The cryoadditives usually operate by increasing the solute concentration in cells. However, in order to be suitable for biological use they must easily penetrate and must not be toxic to cells. Such additives are thus suitable to provide milder storing conditions for the pentamers and/or VLP. Cryoadditives can be supplemented to a composition comprising pentamers and/or VLP to be frozen for storage.

According to a particularly preferred embodiment of the present invention, the cryoadditive is added to the composition comprising VLP for subsequent freezing after the VLP, preferably rVLP, were assembled using two dialysis steps (two-step reassembly).

Suitable molar concentrations of cryoadditives except for the polyol-based cryoadditives may be 0.5 M, 0.6 M, 0.7 M, 0.8 M, 0.9 M, 1 M, 1.1 M, 1.2, 1.3 M, 1.4 M, 1.5 M, 2 M, 3 M, 4 M, 5 M. Preferentially, these cryoadditives are used at a molar concentration of about 1 M, preferably at a molar concentration of 1 M.

The polyol-based cryoadditive may be used at molar concentrations of at least 0.3 M, at least 0.4 M, at least 0.5 M, at least 0.6 M, at least 0.7 M, at least 0.8 M, at least 0.9 M, at least 1 M, at least 2 M or at least 3 M. Preferably, the polyol-based cryoadditives, preferably glycerol 5 %, may be used at a concentration of about 0.6 M to 0.7 M, more preferably at a concentration of 0.68 M.

Alternatively, the polyol-based cryoadditive may be added based on volume percent of the composition comprising pentamers and/or VLP. Suitable volume percent include 3 % (v/v), 4 % (v/v), 5 % (v/v), 6 % (v/v), 7 % (v/v), 8 % (v/v), 9 % (v/v) or 10 % (v/v). Preferentially, the polyol-based cryoadditive is added at 5 % (v/v).

In a preferred embodiment of the invention, the pentamers of step c) and/or the VLP of step d) are subject to purification. The term “purification” in the context of the present invention refers to isolating or separating the VP1, pentamers or VLP from a complex composition. Possible methods include precipitation, cross flow filtration, ultrafiltration, chromatography, for example a preparative chromatography, preferably size exclusion chromatography, and/or dynamic light scattering (DLS). Vivaspin® is an exemplary ultrafiltration unit which can be used.

The term “chromatography” refers to a method which permits the separation of a mixture of substances by distributing the individual components thereof between a stationary phase and a mobile phase. In particular, chromatography refers to a method of purifying a substance by first binding and enriching the substance of interest to the stationary phase before eluting it in a second step (bind-and-elute mode of chromatography) or by binding impurities to the stationary phase and increasing the purity of the molecule of interest in the flow-through (flow-through mode).

Chromatography can be grouped according to the basis of the interaction of the analyte comprised in the mobile phase with the stationary phase. Preferred types of chromatography according to the invention encompass “reversed phase” chromatography, “ion-exchange” chromatography, “affinity” chromatography, or size exclusion chromatography (SEC). Among ion-exchange chromatography, the purification method can be further separated based on the charge present in the stationary phase into cation-exchange chromatography (CEX), in which the stationary phase has a negative charge, thus retaining positively charged molecules, and anion exchange chromatography (AEX), in which the stationary phase has a positive charge, thus retaining negatively charged molecules.

In particular, chromatography may be used to purify rVLP obtained by the method according to the present invention as an intermediate product. In particular AEX may be used to purify rVLP.

The stationary phase used in AEX can be further described based on the strength of the ionic interaction provided by the exchanging material present in the stationary phase into “strong anion exchangers” and “weak anion exchangers”. The expressions “anion exchanger” or “anion exchange matrix” are synonymous and both refer to natural or artificial substances which can bind anions and can exchange these for anions from a surrounding medium. An anion exchanger carries positive ions and exchanges negatively charged counterions.

The VLP of the invention may be further treated with a nuclease, such as a DNAse or an RNAse, and/or be subjected to sterile filtration. A nuclease treatment is preferably done if a nucleic acid, such as an expression vector or an mRNA or a combination thereof, is used as cargo. Different nucleases are known to the skilled person and include for example benzonase. Nucleases are used to hydrolyse residual DNA or mRNA which has not been associated with the VLP. Methods for sterile filtration include, inter alia diafiltration or ultracentrifugation using filters suitable for the removal of impurities. These treatments are especially advantageous for the clinical application of the inventive VLP.

The particle size distribution of a composition comprising VLP according to the invention can be assessed as the “Poly Dispersity Index” (PDI). The PDI indicates the distribution of particle sizes in a composition, and thus describes the uniformity of particles. PDI values can be obtained using different methods, including gel permeation chromatography/size exclusion chromatography, rheology, solution viscosity, membrane osmosis or light scattering.

The PDI preferably is determined by dynamic light scattering (DLS). In DLS, the native distribution is the intensity distribution which indicates how much light is scattered from the various “slices”. DLS allows determination of the mean size and the standard deviation from this mean size from the statistics of the distribution. Relative polydispersity can be determined by dividing the standard deviation by the mean. From the relative polydispersity of a distribution the polydispersity index (PDI) can be derived as its square. PDI values obtained by DLS can be grouped into monodispersed (PDI < 0.1) compositions and polydispersed (PDI > 0.1) compositions, whereby also within the polydispersed group smaller values indicate a more uniform distribution within the composition.

According to the invention, PDI values of 0.1 to 0.4 are preferred. More preferably, the PDI value is 0.1 to 0.3 and even more preferably 0.1 to 0.2.

The average diameter of a composition comprising VLP according to the invention may be measured by visual methods, such as microscopy, preferably equipped with software to determine the average diameter, but also by analytic light scattering methods, such as DLS or nanotracking method, such as NTA.

The VLP content within the composition can be measured by, for example, FFF-MALS and/ or DLS. Both methods can distinguish between the right-sized VLP and aggregates, “tinies” (small particles) and other impurities, such as salts, debris or pentamers.

Such a composition comprising VLP according to the invention in particular fulfills requirements usually imposed on a drug delivery system. Obviously, such a composition is homogeneous and has a high purity.

In another aspect, the invention relates to a drug delivery system obtainable by the method according to the invention. Such a drug delivery system has the advantages as stated supra. In particular, such a drug delivery system can be used in a method of therapy and/or diagnosis, preferably for the treatment of neurological disorders, i.e. CNS diseases, namely a leukodystrophy, in particular Canavan disease or Krabbe disease.

Hence the invention also relates to a method of treating a disorder, in particular a CNS disease, namely a leukodystrophy, in particular Canavan disease or Krabbe disease, with the drug delivery system according to the invention. The method of treating preferably comprises the step of administering the drug delivery system to a subject in need thereof.

The invention also relates to the use of the drug delivery system for the manufacture of a medicament for the treatment of neurological disorders, i.e. CNS diseases, namely a leukodystrophy, in particular Canavan disease or Krabbe disease. The method of treatment preferably does not comprise a step of increasing the permeability of the BBB of the subject to be treated. The drug delivery system of the invention, preferably, is administered to a patient who has not received any chemical or physical treatment for impairing or disrupting the BBB.

In one embodiment, the VLP according to the invention cross the blood brain barrier (BBB). Therefore, in one embodiment of the invention, the drug delivery system may be used to deliver aspartoacylase or galactocerebrosidase, respectively, or the expression vector encoding the respective enzyme or the mRNA encoding the respective enzyme or a combination thereof across the BBB. According to the invention, a drug delivery system and/or a VLP and/or aspartoacylase or galactocerebrosidase, respectively and/or an expression vector encoding the respective enzyme and/or an mRNA encoding the respective enzyme and/or a combination thereof may cross the BBB.

Importantly, the crossing of the BBB by the drug delivery system enables the drug delivery system to exhibit its function of targeting specific cell populations within the brain, i.e. deliver a cargo to targeted cells. In the context of the invention said drug delivery system comprises a delivery to and/or into the targeted cells.

In a preferred embodiment, the VLP of the invention and/or its cargo, after administration to the subject to be treated, in particular a human, can be detected in the CNS in less than 10 days, preferably in less than 5 days, more preferably in less than 3 days after administration. The drug delivery system preferably is administered to the subject intravenously. This is particularly advantageous when using the VLP as a reliable drug delivery system.

Preferably, the method does not require a loss of integrity or increased permeability of the BBB.

According to the invention, it is not required to impair the permeability of the BBB prior or while administering the drug delivery system. Thus, the BBB is preferably physiologically intact which means that the integrity is not decreased and/or the permeability not increased compared with the healthy, native state. The VLP of the invention preferably cross the physiologically intact BBB.

The composition comprising the drug delivery system preferably does not require an additive that may disrupt the integrity of the BBB. Hence, in a most preferred embodiment of the invention the drug delivery system is free of any additive that can impact the permeability of the BBB.

### Materials and Methods

### VLP Manufacturing

Virus-like particles (VLP) were manufactured by protein expression using a Sf9 insect cell line derived from the fall armyworm (Spodoptera frugiperda) (Thermo Fisher Scientific). VLP were produced by infecting the cells with recombinant Baculovirus containing a John Cunningham virus VP1-protein expression cassette. The recombinant Baculovirus was prepared by using the Bac-to-Bac® Baculovirus expression system (Thermo Fisher Scientific). VLP were produced at pH 6.3 after 7 to 10 days in a 3.4 l bioreactor (INFORS HT Minifors). Air flow and temperature (26° C.) were controlled over the time. To remove cells and cell debris suspension was centrifuged at 4° C., 5.000 g and the supernatant containing VLP was harvested.

After that VLP were concentrated using two different concentration methods: precipitation with 7.5 % polyethylenglycol (PEG) or cross flow with an ÄKTAcross flow™ system (GE Healthcare). For PEG precipitation the clarified supernatant was mixed with PEG to achieve 7.5% (v/v) and incubated for 2 h at 4° C., after that the precipitate was separated by centrifugation at 4° C., 10.000 g and suspended in 50 mM NaCl, 10 mM Tris-HCl, pH 7.5. Cross flow was performed with an ÄKTAcross flow™ system equipped with a 300 kDa cut-off membrane (Hydrosart® 300 kDa ECO, Sartorius). The flow ultrafiltration was carried out with a constant pressure of 1.5 bar and a factor of 8 (1 l supernatant against 8 l buffer).

VLP were further dissociated to pentamers by using 5 mM DTT and 10 mM EDTA for 70 min at room temperature and the pentamers purified by anion exchange chromatography (AEX) using HiScale CaptoQ column (GE Healthcare) with a NaCl step gradient from 150 mM to 1 M NaCl. Pentamers were eluted with a 250 mM NaCl step. After elution the pentamers were processed as follows:

Immediately placed into dialysis cassettes with 20 kDa cut-off (Slide-A-Lyzer™ G2 Dialysis Device, Thermo Fisher Scientific) and reassembled by two-step-reassembly by dialysis (two-step dialysis). First, pentamers were dialyzed against 2 M ammonium sulfate buffer (“AS”, 10 mM Tris-HCl, 150 mM NaCl, 2 M (NH4)2SO4, pH 7.5) for 24 h and then transferred for the next 24 h into 10 mM Tris-HCl, 150 mM NaCl, 2 mM CaCl2, pH 7.5 (standard reassociation buffer, “ST”). To separate not reassembled material and aggregates from the VLP, the composition comprising the VLP was purified by size exclusion chromatography (SEC) using HiPrep™ Sephacryl® S-500 HR column (GE Healthcare) under control of polydispersity index (PDI) of fractions in dynamic light scattering (DLS) using Zetasizer ZS Nano (Malvern Inc.). VLP fraction with targeted size were selected and pooled, and then concentrated in Vivaspin® concentrators (Sartorius) with 5 kDa cut-off membrane, if applicable followed by storage at -80° C.

### Artificial Blood-brain-barrier (BBB) Model for Verifying BBB Permeation

BBB permeation of VLP was assessed by using a co-culture in a two-compartment artificial BBB model by direct quantification of expressed hASPA or hGALC mRNA or protein of permeated material (pNL-BB-hASPA or pNL-BB-hGALC packed into VLP) in target cells.

For that purpose, 3*10⁴ of human astrocytoma cells were seeded in a 24-well plate (Greiner Bio-One) filled with 900 µl medium (DMEM + 10% FCS + 1 % Pen/Strep, Thermo Fisher Scientific). 7.5*10⁴ BBB cells (HBEC-5i, cerebral microvascular endothelium) were seeded into 24-well permeable supports (insert, 1 µm, ThinCert, Greiner Bio-One) with 200 µl medium ((DMEM/F-12, HEPES, Thermo Fisher Scientific). Subsequently, inserts were transferred to the 24-well plate in which the human astrocytoma cells were seeded. Inserts without seeded BBB cells were included as control. Cells were incubated for 96 h to 120 h with exchanging the media in the wells every second day.

For BBB permeation assay, 25 µg of loaded VLP with hASPA or hGALC plasmid or hASPA or hGALC plasmid alone were added to the inserts. Samples were also added to inserts without seeded BBB cells as a control. After either 24 h or 48 h the prepared inserts were carefully transferred to a fresh 24-well plate filled with 900 µl of medium (DMEM/F-12, HEPES, Thermo Fisher Scientific) per well and incubated for another 24 h or 48 h. After a total of 72 h the cell pellets were harvested. Insert membranes were cut out after washing with PBS and transferred to a 1.5 ml tube. After detaching the cells with TrypLE, the membranes were removed and the cells were pelleted. The cell pellets were frozen at -80° C. until further usage for RNA isolation, cDNA synthesis and qPCR analysis.

### Cloning of an Expression Vector Encoding hASPA or hGALC

For generating the construct pNL-BB-hASPA, the hASPA gene was synthesized at Thermo Fisher Scientific (GeneArt gene synthesis) as disclosed in SEQ ID NO: 14 of EP2862860 A1 with additionally attached restriction sites on both ends. The gene was inserted into a pNL1.1 plasmid backbone (Promega) with restriction enzyme-based cloning. The construct comprises a CMV promoter.

For generating the construct pNL-BB-hGALC, the hGALC gene was synthesized at Thermo Fisher Scientific (GeneArt gene synthesis) as disclosed in SEQ ID NO: 1 of EP2882284 A1 with addition of the signal sequence comprising 66 nucleotides (see SEQ ID NO: 4 of the instant application and NCBI Reference Sequence NM_000153.4 (nucleotides 1 to 2100)), resulting in the protein sequence as disclosed in SEQ ID NO: 2 of EP2882284 A1 and SEQ ID NO: 3 of the instant application. Restriction sites were attached on both ends. The gene was inserted into a pNL1.1. plasmid backbone (Promega) with restriction enzyme-based cloning. The construct comprises a CMV promoter.

### Packaging of VLP With the Expression Vector Encoding hASPA or hGALC or with mRNA Encoding hASPA or hGALC

For packaging hASPA or hGALC expression vectors or hASPA or hGALC mRNA into VLP, pre-reassembled VLPs were taken from -80° C. and thawed using a thermal shaker (23° C., 350 rpm). Subsequently, VLPs were dissociated by incubating the samples for 15 min at 23° C. and 450 rpm in the presence of dissociation buffer (20 mM Tris-HCl, 150 mM NaCl, 5 mM DTT, and 10 mM EDTA). Dissociated VLP were reassembled in the presence of the hASPA or hGALC expression vector or the hASPA or hGALC mRNA. In short, the dissociated VLP were mixed thoroughly with the hASPA or hGALC expression vector or the hASPA or hGALC mRNA in appropriate concentrations (packaging ratio VLP to expression construct 1:0.2 or 1 :0.5; packaging ratio VLP to mRNA 1 :0.2) followed by dialyzing the mixture against standard reassociation buffer (10 mM Tris-HCl, 150 mM NaCl, 2 mM CaCl2, pH7.5) using a 20 kDa MWCO dialysis chamber (Slide-A-Lyzer™ MINI Dialysis Device, Thermo Fisher Scientific). Samples were incubated for 16 to 18 h. Samples were transferred from the dialysis chamber to a fresh reaction cup and the unpacked nucleic acids, i.e. expression vectors or mRNA, were digested by incubation with 40 u Benzonase® Nuclease (Merck) per 25 µg VLP and 2.5 mM MgCl2 at 37° C. for 1 h. Samples were filtrated (Corning® Costar® Spin-X® centrifuge tube filters; pore size 0.22 µm) and frozen in liquid N2. Afterwards, VLP were analyzed and/or stored at -80° C.

### VLP Characterization

For verifying sample stability, inflection temperatures for each sample were assessed by nDSF using a Tycho NT.6 (Nanotemper) according to the manufacture’s instructions.

To analyze sample composition, asymmetric flow field flow fractionation (AF4, Wyatt Inc.) was performed. Samples were analyzed by using multiangle light scattering (MALS), dynamic light scattering (DLS) and UV detector.

Transmission electron microscopy (TEM) analyses were carried out for directly visualizing the sample at different experimental steps with help of a Zeiss EM900 electron microscope, operating at a voltage of 80 kV. For this approach, samples were stained on carbon-coated copper grids (Plano GmbH) using 2 % uranyl acetate (Sigma Aldrich) beforehand.

For quantifying encapsulated cargo amount, RiboGreen™ Assay was performed in a 96 well plate according to the manufacturer’s instructions (Thermo Fisher Scientific). Fluorescence was measured at 480/520 nm. Encapsulated amount was calculated using calibration curve of the cargo alone.

For verifying the presence of VLP, size-exclusion chromatography (SEC) was performed with help of a Hitachi Chromaster and a Sepax 2000 column with connected pre-column. As running buffer, 10 mM Tris-HCl/150 mM NaCl was used. A diode array detector set to 220 nm, 260 nm, and 280 nm was used for sample analysis.

For analyzing presence of VLP and verifying co-localization of protein and encapsulated cargo, agarose gel electrophoresis (AGE) was performed using 1 % agarose gel and Tris-acetate buffer. Loading buffer was prepared using glycerol and bromphenol blue. Encapsulated cargo was visualized after incubating the gel with GelRed™. To visualize VLPs, gels were incubated with Instant Blue. Images were taken using Biorad Gel Doc™ XR+ Gel Documentation System.

### hASPA or hGALC Expression in Cells: RNA Isolation, cDNA Synthesis and qPCR

### Cell Treatment With hASPA or hGALC Expression Vectors

After incubating the different cell lines for 48 or 72 h with VLP packed with a hASPA- or hGALC-encoding plasmid or the hASPA- or hGALC-encoding plasmid alone, human astrocytoma or mouse fibroblast cells were harvested using trypsin, pelleted, and washed using PBS. Cells transfected with the hASPA- or hGALC-encoding plasmid were also harvested and pelleted for subsequent RNA isolation, cDNA synthesis and qPCR. For this, Lipofectamin 3000 (Thermo Scientific) was incubated with Opti-MEM (Thermo Scientific) and 1 µg of plasmid DNA was incubated with P3000 reagent (Thermo Scientific), mixed and incubated for 15 min at room temperature and added to the cells. Cells were incubated for 48 h at 37° C.

### Cell Treatment With hASPA or hGALC mRNAs

After incubating 30.000 cells for 24 h with VLP packed with hASPA or hGALC encoding mRNA or the hASPA/hGALC encoding mRNA alone, human astrocytoma cells were harvested using trypsin, pelleted, and washed using PBS. Cells transfected with the hASPA/hGALC encoding mRNA were also harvested and pelleted for subsequent RNA isolation, cDNA synthesis and qPCR. For transfection, diluted transfection reagent solution (Stemfect™ transfection kit) and diluted mRNA solution were mixed and incubated for 15 min at room temperature and added to the cells. Incubation with 6.25 µg VLP, 10 ng mRNA or transfection with 10 ng mRNA, respectively. Cells were incubated for 24 h at 37° C. Two wells were pooled for RNA isolation/cDNA synthesis.

### RNA Isolation

The RNA was isolated from the cell pellet using an RNA Isolation Kit (Macherey-Nagel). After Isolation, RNA concentration was determined using NanoDrop (Thermo Scientific).

### cDNA Synthesis

For cDNA synthesis between 250 ng and 500 ng (expression vectors) or 400 ng (mRNA) RNA were used. cDNA was synthesized with the RevertAid First Strand cDNA synthesis kit (Thermo Scientific) using a primer mix composed of Oligo-DT primer and random primer in a ratio of 1:2. A master mix of primer mix, dNTPs, reaction buffer, RiboLock and RevertAid was added to the mRNA/water mixture and the synthesis was performed in one step in the thermocycler. Afterwards, cDNA samples were diluted 1:5 in ddH₂O and samples were stored at -20° C. until use.

### qPCR

qPCR was performed in a Lightcycler (CFX96 Touch, BioRad) using EvaGreen Dye and the appropriate primers for hASPA or hGALC in a 1:20 dilution.

### Immunocytochemistry

After incubating 30.000 cells on coverslips for 48 h with 6.25 µg VLP packed with hASPA or hGALC encoding mRNA or 10 ng of hASPA/hGALC encoding mRNA alone, human astrocytoma cells were washed with PBS and fixed with 4% PFA. Cells transfected with 10 ng of the hASPA/hGALC encoding mRNA were also washed with PBS and fixed with 4% PFA. Cells were incubated with lipofection as described supra for 48 h at 37° C.

Fixed cells were washed with PBS, permeabilized with 0.2% Triton-X-100, again washed and blocked with 1% BSA for 30 min at room temperature. Cells were incubated with the primary antibody (GALC: Poteintech; ASPA: Abcam) overnight at 4° C.

Cells were incubated with the secondary antibody diluted in 1% BSA (goat anti rabbit IgG Cy5-labelled, Abcam) for 2 h at room temperature. Cells were washed with PBS and covered with RotiMount (with DAPI, Roth) on a microscope slide. Dried samples were analyzed with a confocal microscope (Leica SP8).

### hASPA and hGALC Expression in Mice Brain

Balb/C mice (4 per group per construct per timepoint) were injected with VLP associated with hASPA or hGALC mRNA or injected with mRNA alone into tail vein and terminated 6 or 24 h after injection. Mice were perfused with PBS and brain was taken out and shock frozen.

One thawed mouse brain hemisphere per animal (n=4 per group) was lysed using the GentleMACS™ Dissociator (Miltenyi Biotec) and RNA isolation was performed according to the instructions of the NucleoSpin® RNA Isolation Kit (Macherey-Nagel).

cDNA Synthesis was performed using the RevertAid™ First strand cDNA synthesis kit (Thermo Fisher Scientific) according to the manufacturer’s instructions.

qPCR was performed as described supra (hASPA or hGALC expression in cells). For the qPCR with brain samples Biorad Opus 384 Thermal Cycler was used and fold-change in comparison to buffer control was calculated.

### EXAMPLES

### 1. Detection of ASPA Expression in Mouse Organs

To ensure adequate and notably specific detection of human ASPA expression in subsequent experiments, endogenous expression of mouse ASPA was assessed in the different mouse organs using species specific primers. The results are depicted in FIG. 1. It can be seen that when mouse specific primers are used, ASPA expression is detected in all organs, with the highest levels (corresponding to the lowest value) in the kidney and the lung. In contrast, with primers specific for human ASPA, no ASPA expression is detected in mouse organs, thus demonstrating the suitability for the primers to specifically detect expression of human ASPA (hASPA).

### 2. hASPA Expression and Packaging of VLP With the Expression Vector Encoding hASPA

VLP were mixed with a hASPA-encoding plasmid in packaging ratios VLP to expression construct of 1:0.2 and 1:0.5. The thus packaged VLP were characterized as described in Materials and Methods supra. Human astrocytoma cells and mouse fibroblasts were incubated with the packaged VLP and hASPA expression was determined. Cells transfected with the hASPA-encoding plasmid and cells incubated with unpackaged plasmid served as controls. The results are summarized in Table 4 and depicted in FIG. 2.

TABLE 4

| Sample | Ti °C (nDSF) | PDI {z=56-62 nm} | % EnPCs (AF4) | hASPA expression (Fold-Change, qPCR) | Agarose gel electrophoresis sharp bands |
|---|---|---|---|---|---|
| pNL-BB-hASPA 1:0.2 | 72.3 | 0.184 | 69.4 | 1575 | + |
| pNL-BB-hASPA 1:0.5 | 72 | 0.170 | 73.4 | 2051 | + |

### 3. VLP Associated with a hASPA Plasmid Lead to hASPA Expression in Astrocytomas in an in Vitro BBB Model

BBB permeation of VLP was assessed as described in Materials and Methods supra. The results are depicted in FIG. 3. hASPA is notably expressed in astrocytoma cells that have been co-cultured with BBB cells (HBEC-5i endothelial cells) demonstrating that the VLP associated with hASPA crosses the artificial BBB. Longer co-culture leads to higher expression in astrocytoma cells that have been co-cultured with BBB cells, indicating increased permeation of VLP associated with hASPA through the artificial BBB with time and an increased expression of hASPA in astrocytoma cells and a decrease in endothelial cells with time.

### 4. Detection of GALC Expression in Mouse Organs

To ensure adequate and notably specific detection of human GALC expression in subsequent experiments, endogenous expression of mouse GALC was assessed in the different mouse organs using species specific primers. The results are depicted in FIG. 4. It can be seen that when mouse specific primers are used, GALC expression is detected in all organs, with the highest levels (corresponding to the lowest value) in the kidney and the lung. In contrast, with primers specific for human GALC, no GALC expression is detected in mouse organs, thus demonstrating the suitability for the primers to specifically detect expression of human GALC (hGALC).

### 5. hGALC Expression and Packaging of VLP with the Expression Vector Encoding hGALC

VLP were mixed with a hGALC-encoding plasmid in packaging ratios VLP to expression construct of 1 :0.2. The thus packaged VLP were characterized as described in Materials and Methods supra. Human astrocytoma cells and mouse fibroblasts were incubated with the packaged VLP and hGALC expression was determined. The results are summarized in Table 5 and depicted in FIG. 5.

TABLE 5

| Sample | Ti °C (nDSF) | PDI {z=56-62 nm} | % EnPCs (AF4) | hASPA expression (Fold-Change, qPCR) | Agarose gel electrophoresis sharp bands |
|---|---|---|---|---|---|
| pNL-BB-hGALC 1:0.2 | 71.7 | 0.04 | 93.4 | 2191 | + |

### 6. VLP Associated with a hGALC Plasmid Lead to hGALC Expression in Astrocytomas in an in Vitro BBB Model

BBB permeation of VLP was assessed as described in Materials and Methods supra. The results are depicted in FIG. 6. hGALC is notably expressed in astrocytoma cells that have been co-cultured with BBB cells (HBEC-5i endothelial cells) demonstrating that the VLP associated with hGALC crosses the artificial BBB. Longer co-culture leads to higher expression in astrocytoma cells that have been co-cultured with BBB cells, indicating increased permeation of VLP associated with hGALC through the artificial BBB with time and an increased expression of hGALC in astrocytoma cells and a decrease of hGALC in endothelial cells with time.

### 7. Packaging of VLP With hASPA and hGALC mRNA

Packaging of VLP with mRNA encoding the enzymes hASPA and hGALC was performed as described supra. An overview is given in Table 6:

TABLE 6

| | | |
|---|---|---|
| Assay | hASPA mRNA | hGALC mRNA |
| Ti °C (nDSF) | 71 | 71 |
| RiboGreen [ng mRNA/µg VLP] | 20 | 20 |
| SEC (VLPs in sample) | + | + |
| AF4 (VLP >45% of sample) | + | + |
| TEM (VLPs visible in TEM) | + | + |
| Agarose Gel Electrophoresis [mRNA-associated VLP Band] | + | + |

As can be taken from Table 6, VLP packed with hASPA or hGALC mRNA are stable and can be packed with mRNA. Uniformly sized VLP in the sample after packaging can be detected with different methods. An mRNA-associated VLP band is also visible (agarose gel electrophoresis). Hence, packaging with both RNA was effective.

Both VLP (associated with hASPA or hGALC mRNA, respectively) show a uniform size distribution, i. e. only one peak in DLS analyses (FIG. 7).

### 8. hASPA and hGALC Expression in Cells (mRNA as Cargo) / qPCR

To evaluate expression of hASPA and hGALC mRNA in human astrocytoma cells after using mRNA as cargo of VLP, qPCR analyses were performed as described supra. After incubation with VLP associated with the mRNA, high amounts of both mRNA were detected compared with both controls (FIG. 8). Thus, it was shown that VLP associated with hASPA and hGALC mRNA can be effectively used for cell transfection.

### 9. hASPA and hGALC Expression in Cells (mRNA as Cargo) / Immunocytochemistry

hASPA and hGALC expression in human astrocytoma cells was confirmed by immunocytochemistry (FIG. 9). Both hASPA and hGALC protein expression was detected in the samples after incubation of cells with VLP associated with the mRNA. Much less or no expression is visible in the control samples.

### 10. hASPA and hGALC Expression in Mice Brain

In vivo experiments with lysed mice brains confirmed hASPA and hGALC expression (mRNA) after injection of VLP associated with the mRNA into mice (FIG. 10). The controls show a much lower expression. Expression is higher after 6 hours compared with 24 hours, possibly due to mRNA usage and depletion over time.

### EMBODIMENTS

1. VLP associated with an enzyme or an expression vector encoding said enzyme for use in a method for the treatment of a leukodystrophy in a subject, wherein the enzyme is aspartoacylase or galactocerebrosidase.

2. The VLP for use according to embodiment 1, wherein
(i) the enzyme is aspartoacylase and the leukodystrophy is Canavan disease; or
(ii) the enzyme is galactocerebrosidase and the leukodystrophy is Krabbe disease.

3. The VLP for use according to embodiment 1 or 2, wherein the VLP does not comprise viral genetic material and the expression vector does not encode viral proteins.

4. The VLP for use according to any of the preceding embodiments, wherein the subject is an animal or a human being, preferably a human being.

5. The VLP for use according to any of the preceding embodiments, wherein the enzyme comprises an amino acid sequence which is at least 80 %, preferably at least 90 % identical to the amino acid sequence according to SEQ ID NO: 1 over its entire length, more preferably has the amino acid sequence of SEQ ID NO: 1; or wherein the enzyme comprises an amino acid sequence which is at least 80 %, preferably at least 90 % identical to the amino acid sequence according to SEQ ID NO: 3 over its entire length, more preferably has the amino acid sequence of SEQ ID NO: 3.

6. The VLP for use according to any of the preceding embodiments, wherein the expression vector has a size of less than 7 kb, preferably less than 6 kb, more preferably less than 5 kb, most preferably less than 4 kb.

7. The VLP for use according to any of the preceding embodiments, wherein the expression vector has a promoter selected from the group consisting of CMV and CAG.

8. The VLP for use according to any of the preceding embodiments, wherein the enzyme is encoded by a nucleotide sequence which is at least 70 %, preferably at least 80 %, more preferably at least 90 % identical to the nucleotide sequence of SEQ ID NO: 2 over its entire length, most preferably is the nucleotide sequence of SEQ ID NO: 2; or the enzyme is encoded by a nucleotide sequence which is at least 70 %, preferably at least 80 %, more preferably at least 90 % identical to the nucleotide sequence of SEQ ID NO: 4 over its entire length, most preferably is the nucleotide sequence of SEQ ID NO: 4.

9. The VLP for use according to any of the preceding embodiments, wherein the VLP is derived from a human polyoma virus, preferably JCV.

10. The VLP for use according to any of the preceding embodiments, wherein the VLP crosses the blood-brain barrier, preferably the physiologically intact blood-brain barrier, to enter the CNS together with the enzyme or the expression vector.

11. The VLP according to embodiment 10, wherein the enzyme or the expression vector enters astrocytes, oligodendrocytes, microglia or neurons, preferably oligodendrocytes.

12. The VLP for use according to any of the preceding embodiments, wherein the VLP is administered orally or parenterally, preferably intravenously.

13. The VLP for use according to any of the preceding embodiments, wherein a target cell is contacted with an effective amount of the enzyme.

14. The VLP for use according to any of the preceding embodiments, wherein the enzyme has a therapeutically effective enzyme activity for at least 10 days, preferably for at least 20 days, more preferably for at least 30 days.

15. The VLP for use according to any of the preceding embodiments, wherein the VLP is composed of VP1 proteins of JC virus.

16. The VLP according to embodiment 15, wherein the VP1 protein comprises an amino acid sequence which is at least 80 % identical to the amino acid sequence according to SEQ ID NO: 5 or 6 over its entire length, preferably at least 90 % identical.

17. Pharmaceutical composition for use in a method for the treatment of a leukodystrophy in a subject, wherein the pharmaceutical composition comprises the VLP according to any of embodiments 1 to 16 and a pharmaceutically acceptable carrier, and/or excipient.

18. Expression vector having a coding region encoding an enzyme, a promoter selected from the group consisting of CAG and CMV, and having a size of less than 7 kb, preferably less than 6 kb, more preferably less than 5 kb, most preferably less than 4 kb, wherein the enzyme is aspartoacylase or galactocerebrosidase.

19. The expression vector according to embodiment 18, wherein the coding region comprises a nucleotide sequence which is at least 70 %, preferably at least 80 %, more preferably at least 90 % identical to the nucleotide sequence of SEQ ID NO: 2 over its entire length, most preferably is the nucleotide sequence of SEQ ID NO: 2, or wherein the coding region comprises a nucleotide sequence which is at least 70 %, preferably at least 80 %, more preferably at least 90 % identical to the nucleotide sequence of SEQ ID NO: 4 over its entire length, most preferably is the nucleotide sequence of SEQ ID NO: 4.

20. Method of associating a VLP with an enzyme, or an expression vector encoding an enzyme, wherein the enzyme is aspartoacylase or galactocerebrosidase, and wherein the method comprises the following steps:
a) providing a composition comprising VP1 proteins,
b) exposing the VP1 proteins of the composition of a) to conditions inducing the VP1 to assemble into VLP,
c) exposing the VLP of the composition of b) to conditions disassembling the VLP into pentamers,
d) exposing the pentamers of the composition of c) to conditions inducing the pentamers to reassemble into VLP
e) exposing the VLP of the composition of d) to conditions disassembling the VLP into pentamers,
f) exposing the pentamers of the composition of e) to the enzyme or the expression vector to conditions inducing the pentamers to assemble into a VLP associated with the enzyme or the expression vector.

21. VLP obtainable by the method according to embodiment 20.

1. VLP associated with an enzyme or an expression vector encoding said enzyme or an mRNA encoding said enzyme or a combination thereof for use in a method for the treatment of a leukodystrophy in a subject, wherein the enzyme is aspartoacylase or galactocerebrosidase.

2. The VLP for use according to claim 1, wherein
(i) the enzyme is aspartoacylase and the leukodystrophy is Canavan disease; or
(ii)the enzyme is galactocerebrosidase and the leukodystrophy is Krabbe disease.

3. The VLP for use according to claim 1, wherein the VLP does not comprise viral genetic material and the expression vector or the mRNA does not encode viral proteins.

4. The VLP for use according to claim 1, wherein the subject is an animal or a human being, preferably a human being.

5. The VLP for use according to claim 1, wherein the enzyme comprises an amino acid sequence which is at least 80 %, preferably at least 90 % identical to the amino acid sequence according to SEQ ID NO: 1 over its entire length, more preferably has the amino acid sequence of SEQ ID NO: 1; or wherein the enzyme comprises an amino acid sequence which is at least 80 %, preferably at least 90 % identical to the amino acid sequence according to SEQ ID NO: 3 over its entire length, more preferably has the amino acid sequence of SEQ ID NO: 3.

6. The VLP for use according to claim 1, wherein the expression vector has a size of less than 7 kb, preferably less than 6 kb, more preferably less than 5 kb, most preferably less than 4 kb.

7. The VLP for use according to claim 1, wherein the expression vector has a promoter selected from the group consisting of CMV and CAG.

8. The VLP for use according to claim 1, wherein the enzyme is encoded by a nucleotide sequence which is at least 70 %, preferably at least 80 %, more preferably at least 90 % identical to the nucleotide sequence of SEQ ID NO: 2 over its entire length, most preferably is the nucleotide sequence of SEQ ID NO: 2; or the enzyme is encoded by a nucleotide sequence which is at least 70 %, preferably at least 80 %, more preferably at least 90 % identical to the nucleotide sequence of SEQ ID NO: 4 over its entire length, most preferably is the nucleotide sequence of SEQ ID NO: 4.

9. The VLP for use according to claim 1, wherein the enzyme is encoded by an mRNA sequence which is at least 70 %, preferably at least 80 %, more preferably at least 90 % identical to the mRNA sequence of SEQ ID NO: 9 over its entire length, most preferably comprises the mRNA sequence of SEQ ID NO: 9; or the enzyme is encoded by an mRNA sequence which is at least 70 %, preferably at least 80 %, more preferably at least 90 % identical to the mRNA sequence of SEQ ID NO: 10 over its entire length, most preferably comprises the mRNA sequence of SEQ ID NO: 10.

10. The VLP for use according to claim 1, wherein the VLP is derived from a human polyoma virus, preferably JCV.

11. The VLP for use according to claim 1, wherein the VLP crosses the blood-brain barrier, preferably the physiologically intact blood-brain barrier, to enter the CNS together with the enzyme or the expression vector or the mRNA or the combination thereof.

12. The VLP according to claim 11, wherein the enzyme or the expression vector or the mRNA or the combination thereof enters astrocytes, oligodendrocytes, microglia or neurons, preferably oligodendrocytes.

13. The VLP for use according to claim 1, wherein the VLP is administered orally or parenterally, preferably intravenously.

14. The VLP for use according to claim 1, wherein a target cell is contacted with an effective amount of the enzyme.

15. The VLP for use according to claim 1, wherein the enzyme has a therapeutically effective enzyme activity for at least 10 days, preferably for at least 20 days, more preferably for at least 30 days.

16. The VLP for use according to claim 1, wherein the VLP is composed of VP1 proteins of JC virus.

17. The VLP according to claim 16, wherein the VP1 protein comprises an amino acid sequence which is at least 80 % identical to the amino acid sequence according to SEQ ID NO: 5 or 6 over its entire length, preferably at least 90 % identical.

18. Pharmaceutical composition for use in a method for the treatment of a leukodystrophy in a subject, wherein the pharmaceutical composition comprises the VLP according to claim 1 and a pharmaceutically acceptable carrier, and/or excipient.

19. Expression vector having a coding region encoding an enzyme, a promoter selected from the group consisting of CAG and CMV, and having a size of less than 7 kb, preferably less than 6 kb, more preferably less than 5 kb, most preferably less than 4 kb, wherein the enzyme is aspartoacylase or galactocerebrosidase.

20. The expression vector according to claim 19, wherein the coding region comprises a nucleotide sequence which is at least 70 %, preferably at least 80 %, more preferably at least 90 % identical to the nucleotide sequence of SEQ ID NO: 2 over its entire length, most preferably is the nucleotide sequence of SEQ ID NO: 2, or wherein the coding region comprises a nucleotide sequence which is at least 70 %, preferably at least 80 %, more preferably at least 90 % identical to the nucleotide sequence of SEQ ID NO: 4 over its entire length, most preferably is the nucleotide sequence of SEQ ID NO: 4.

21. Method of associating a VLP with an enzyme, or an expression vector encoding an enzyme or an mRNA encoding an enzyme or a combination thereof, wherein the enzyme is aspartoacylase or galactocerebrosidase, and wherein the method comprises the following steps:
a) providing a composition comprising VP1 proteins,
b) exposing the VP1 proteins of the composition of a) to conditions inducing the VP1 to assemble into VLP,
c) exposing the VLP of the composition of b) to conditions disassembling the VLP into pentamers,
d) exposing the pentamers of the composition of c) to conditions inducing the pentamers to reassemble into VLP
e) exposing the VLP of the composition of d) to conditions disassembling the VLP into pentamers,
f) exposing the pentamers of the composition of e) to the enzyme or the expression vector or the mRNA or the combination thereof to conditions inducing the pentamers to assemble into a VLP associated with the enzyme or the expression vector or the mRNA or the combination thereof.

22. VLP obtainable by the method according to claim 21.


SEQUENCE LISTING (10 sequences)

SEQ ID NO: 1 - protein, 313 aa, Homo sapiens
MTSCHIAEEHIQKVAIFGGTHGNELTGVFLVKHWLENGAEIQRTGLEVKPFITNPRAVKKCTRYIDCDLNRIFDLENLGKKMSEDLPYEVRRAQEINHLFGPKDSEDSYDIIFDLHNTTSNMGCTLILEDSRNNFLIQMFHYIKTSLAPLPCYVYLIEHPSLKYATTRSIAKYPVGIEVGPQPQGVLRADILDQMRKMIKHALDFIHHFNEGKEFPPCAIEVYKIIEKVDYPRDENGEIAAIIHPNLQDQDWKPLHPGDPMFLTLDGKTIPLGGDCTVYPVFVNEAAYYEKKEAFAKTTKLTLNAKSIRCCLH

SEQ ID NO: 2 - DNA, 942 bp, Homo sapiens
atgacttcttgtcacattgctgaagaacatatacaaaaggttgctatctttggaggaacccatgggaatgagctaaccggagtatttctggttaagcattggctagagaatggcgctgagattcagagaacagggctggaggtaaaaccatttattactaaccccagagcagtgaagaagtgtaccagatatattgactgtgacctgaatcgcatttttgaccttgaaaatcttggcaaaaaaatgtcagaagatttgccatatgaagtgagaagggctcaagaaataaatcatttatttggtccaaaagacagtgaagattcctatgacattatttttgaccttcacaacaccacctctaacatggggtgcactcttattcttgaggattccaggaataactttttaattcagatgtttcattacattaagacttctctggctccactaccctgctacgtttatctgattgagcatccttccctcaaatatgcgaccactcgttccatagccaagtatcctgtgggtatagaagttggtcctcagcctcaaggggttctgagagctgatatcttggatcaaatgagaaaaatgattaaacatgctcttgattttatacatcatttcaatgaaggaaaagaatttcctccctgcgccattgaggtctataaaattatagagaaagttgattacccccgggatgaaaatggagaaattgctgctatcatccatcctaatctgcaggatcaagactggaaaccactgcatcctggggatcccatgtttttaactcttgatgggaagacgatcccactgggcggagactgtaccgtgtaccccgtgtttgtgaatgaggccgcatattacgaaaagaaagaagcttttgcaaagacaactaaactaacgctcaatgcaaaaagtattcgctgctgtttacattag

SEQ ID NO: 3 - protein, 685 aa, Homo sapiens
MAEWLLSASWQRRAKAMTAAAGSAGRAAVPLLLCALLAPGGAYVLDDSDGLGREFDGIGAVSGGGATSRLLVNYPEPYRSQILDYLFKPNFGASLHILKVEIGGDGQTTDGTEPSHMHYALDENYFRGYEWWLMKEAKKRNPNITLIGLPWSFPGWLGKGFDWPYVNLQLTAYYVVTWIVGAKRYHDLDIDYIGIWNERSYNANYIKILRKMLNYQGLQRVKIIASDNLWESISASMLLDAELFKVVDVIGAHYPGTHSAKDAKLTGKKLWSSEDFSTLNSDMGAGCWGRILNQNYINGYMTSTIAWNLVASYYEQLPYGRCGLMTAQEPWSGHYVVESPVWVSAHTTQFTQPGWYYLKTVGHLEKGGSYVALTDGLGNLTIIIETMSHKHSKCIRPFLPYFNVSQQFATFVLKGSFSEIPELQVWYTKLGKTSERFLFKQLDSLWLLDSDGSFTLSLHEDELFTLTTLTTGRKGSYPLPPKSQPFPSTYKDDFNVDYPFFSEAPNFADQTGVFEYFTNIEDPGEHHFTLRQVLNQRPITWAADASNTISIIGDYNWTNLTIKCDVYIETPDTGGVFIAGRVNKGGILIRSARGIFFWIFANGSYRVTGDLAGWIIYALGRVEVTAKKWYTLTLTIKGHFTSGMLNDKSLWTDIPVNFPKNGWAAIGTHSFEFAQFDNFLVEATR

SEQ ID NO: 4 - DNA, 2100 bp, Homo sapiens
agtcatgtgacccacacaatggctgagtggctactctcggcttcctggcaacgccgagcgaaagctatgactgcggccgcgggttcggcgggccgcgccgcggtgcccttgctgctgtgtgcgctgctggcgcccggcggcgcgtacgtgctcgacgactccgacgggctgggccgggagttcgacggcatcggcgcggtcagcggcggcggggcaacctcccgacttctagtaaattacccagagccctatcgttctcagatattggattatctctttaagccgaattttggtgcctctttgcatattttaaaagtggaaataggtggtgatgggcagacaacagacggcactgagccctcccacatgcattatgcactagatgagaattatttccgaggatacgagtggtggttgatgaaagaagctaagaagaggaatcccaatattacactcattgggttgccatggtcattccctggatggctgggaaaaggtttcgactggccttatgtcaatcttcagctgactgcctattatgtcgtgacctggattgtgggcgccaagcgttaccatgatttggacattgattatattggaatttggaatgagaggtcatataatgccaattatattaagatattaagaaaaatgctgaattatcaaggtctccagcgagtgaaaatcatagcaagtgataatctctgggagtccatctctgcatccatgctccttgatgccgaactcttcaaggtggttgatgttataggggctcattatcctggaacccattcagcaaaagatgcaaagttgactgggaagaagctttggtcttctgaagactttagcactttaaatagtgacatgggtgcaggctgctggggtcgcattttaaatcagaattatatcaatggctatatgacttccacaatcgcatggaatttagtggctagttactatgaacagttgccttatgggagatgcgggttgatgacggcccaggagccatggagtgggcactacgtggtagaatctcctgtctgggtatcagctcataccactcagtttactcaacctggctggtattacctgaagacagttggccatttagagaaaggaggaagctacgtagctctgactgatggcttagggaacctcaccatcatcattgaaaccatgagtcataaacattctaagtgcatacggccatttcttccttatttcaatgtgtcacaacaatttgccacctttgttcttaagggatcttttagtgaaataccagagctacaggtatggtataccaaacttggaaaaacatccgaaagatttctttttaagcagctggattctctatggctccttgacagcgatggcagtttcacactgagcctgcatgaagatgagctgttcacactcaccactctcaccactggtcgcaaaggcagctacccgcttcctccaaaatcccagcccttcccaagtacctataaggatgatttcaatgttgattacccattttttagtgaagctccaaactttgctgatcaaactggtgtatttgaatattttacaaatattgaagaccctggcgagcatcacttcacgctacgccaagttctcaaccagagacccattacatgggctgccgatgcatccaacacaatcagtattataggagactacaactggaccaatctgactataaagtgtgatgtatacatagagacccctgacacaggaggtgtgttcattgcaggaagagtaaataaaggtggtattttgattagaagtgccagaggaattttcttctggatttttgcaaatggatcttacagggttacaggtgatttagctggatggattatatatgctttaggacgtgttgaagttacagcaaaaaaatggtatacactcacgttaactattaagggtcatttcacctctggcatgctgaatgacaagtctctgtggacagacatccctgtgaattttccaaagaatggctgggctgcaattggaactcactcctttgaatttgcacagtttgacaactttcttgtggaagccacacgctaatacttaacagggcatcatagaata

SEQ ID NO: 5 - protein, 354 aa, Artificial Sequence
  derived from JC virus
MAPTKRKGEPKDPVQVPKLLIRGGVEVLEVKTGVDSITEVECFLTPEMGDPDEHLRGFSKSISISDTFESDSPNRDMLPCYSVARIPLPNLNEDLTCGNILMWEAVTLKTEVIGVTSLMNVHSNGQATHDNGAGKPVQGTSFHFFSVGGEALELQGVVFNYRTKYPDGTIFPKNATVQSQVMNTEHKAYLDKNKAYPVECWVPDPTRNENTRYFGTLTGGENVPPVLHITNTATTVLLDEFGVGPLCKGDNLYLSAVDVCGMFTNRSGSQQWRGLSRYFKVQLRKRRVKNPYPISFLLTDLINRRTPRVDGQPMYGMDAQVEEVRVFEGTEELPGDPDMMRYVDKYGQLQTKML

SEQ ID NO: 6 - protein, 354 aa, JC virus
MAPTKRKGERKDPVQVPKLLIRGGVEVLEVKTGVDSITEVECFLTPEMGDPDEHLRGFSKSISISDTFESDSPSKDMLPCYSVARIPLPNLNEDLTCGNILMWEAVTLKTEVIGVTSLMNVHSNGQAAHDNGAGKPVQGTSFHFFSVGGEALELQGVVFNYRTKYPDGTIFPKNATVQSQVMNTEHKAYLDKNKAYPVECWVPDPTRNENTRYFGTLTGGENVPPVLHITNTATTVLLDEFGVGPLCKGDNLYLSAVDVCGMFTNRSGSQQWRGLSRYFKVQLRKRRVKNPYPISFLLTDLINRRTPRVDGQPMYGMDAQVEEVRVFEGTEELPGDPDMMRYVDRYGQLQTKML

SEQ ID NO: 7 - DNA, 1065 bp, Artificial Sequence
  derived from JC virus
atggctcccaccaagcgcaagggcgagcccaaggaccccgtgcaagtgcccaagctgctgatccgtggtggtgtcgaggtgctggaagtcaagaccggcgtggactccattaccgaggtggagtgcttcctcacccccgagatgggtgaccctgacgagcacctgaggggcttctccaagtccatctccatctccgacaccttcgagtccgactcccccaaccgtgacatgctgccctgctactccgtggctcgtatccccctgcccaacctgaacgaggacctgacttgcggcaacatcctgatgtgggaggctgtgaccctcaagaccgaggtcatcggcgtgacttccctgatgaacgtgcactccaacggccaggctacccacgacaacggtgctggcaagcccgtgcagggaacctccttccacttcttctccgtgggtggcgaggctctggaactccagggcgtggtgttcaactaccgtaccaagtaccccgacggcaccatcttccccaagaacgctactgtgcagtcccaagtgatgaacaccgagcacaaggcttacctggacaagaacaaggcctaccccgtggagtgctgggtgcccgaccccacccgtaacgagaacacccgttacttcggcaccctgaccggtggagagaacgtgccccccgtgctgcacatcaccaacaccgctaccaccgtgctgctggacgagttcggtgtcggtcccctgtgcaagggcgacaacctgtacctgtccgctgtggacgtgtgcggcatgttcaccaaccgttccggttcccagcagtggcgtggcctgtcccgctacttcaaggtgcagctgcgcaagcgtcgtgtgaagaacccctaccctatctccttcctgctgaccgacctgatcaaccgtcgtacccctcgtgtggacggccagcccatgtacggcatggacgctcaggtggaagaggtccgcgtgttcgagggcaccgaggaattgcccggcgaccccgacatgatgcgttacgtggacaagtacggccagctccagaccaagatgctgtaa

SEQ ID NO: 8 - DNA, 1065 bp, JC virus
atggccccaacaaaaagaaaaggagaaaggaaggaccccgtgcaagttccaaaacttcttataagaggaggagtagaagttctagaagttaaaactggggttgactcaattacagaggtagaatgctttttaactccagaaatgggtgacccagatgagcatcttaggggttttagtaagtcaatatctatatcagatacatttgaaagtgactccccaaatagggacatgcttccttgttacagtgtggccagaattccactacccaatctaaatgaggatctaacctgtggaaatatactcatgtgggaggctgtgaccttaaaaactgaggttataggggtgacaagtttgatgaatgtgcactctaatgggcaagcaactcatgacaatggtgcagggaagccagtgcagggcaccagctttcattttttttctgttgggggggaggctttagaattacagggggtgctttttaattacagaacaaagtacccagatggaacaatttttccaaagaatgccacagtgcaatctcaagtcatgaacacagagcacaaggcgtacctagataagaacaaagcatatcctgttgaatgttgggttcctgatcccaccagaaatgaaaacacaagatattttgggacactaacaggaggagaaaatgttcctccagttcttcatataacaaacactgccacaacagtgttgcttgatgaatttggtgttgggccactttgcaaaggtgacaacttatacttgtcagctgttgatgtctgtggcatgtttacaaacaggtctggttcccagcagtggagaggactctccagatattttaaggtgcagctaaggaaaaggagggttaaaaacccctacccaatttctttccttcttactgatttaattaacagaaggactcctagagttgatgggcagcctatgtatggcatggatgctcaagtagaggaggttagagtttttgagggaacagaggagcttccaggggacccagacatgatgagatacgttgacaaatatggacagttgcagacaaaaatgctgtaa

SEQ ID NO: 9 - RNA, 1140 nt, Homo sapiens
aggaaauaagagagaaaagaagaguaagaagaaauauaagagccaccaugacuucuugucacauugcugaagaacauauacaaaagguugcuaucuuuggaggaacccaugggaaugagcuaaccggaguauuucugguuaagcauuggcuagagaauggcgcugagauucagagaacagggcuggagguaaaaccauuuauuacuaaccccagagcagugaagaaguguaccagauauauugacugugaccugaaucgcauuuuugaccuugaaaaucuuggcaaaaaaaugucagaagauuugccauaugaagugagaagggcucaagaaauaaaucauuuauuugguccaaaagacagugaagauuccuaugacauuauuuuugaccuucacaacaccaccucuaacauggggugcacucuuauucuugaggauuccaggaauaacuuuuuaauucagauguuucauuacauuaagacuucucuggcuccacuacccugcuacguuuaucugauugagcauccuucccucaaauaugcgaccacucguuccauagccaaguauccuguggguauagaaguugguccucagccucaagggguucugagagcugauaucuuggaucaaaugagaaaaaugauuaaacaugcucuugauuuuauacaucauuucaaugaaggaaaagaauuuccucccugcgccauugaggucuauaaaauuauagagaaaguugauuacccccgggaugaaaauggagaaauugcugcuaucauccauccuaaucugcaggaucaagacuggaaaccacugcauccuggggaucccauguuuuuaacucuugaugggaagacgaucccacugggcggagacuguaccguguaccccguguuugugaaugaggccgcauauuacgaaaagaaagaagcuuuugcaaagacaacuaaacuaacgcucaaugcaaaaaguauucgcugcuguuuacauuaggcggccgcuuaauuaagcugccuucugcggggcuugccuucuggccaugcccuucuucucucccuugcaccuguaccucuuggucuuugaauaaagccugaguaggaagucuagaguuuaaacauuuaaaucugcagaucccaauggcgcg

SEQ ID NO: 10 - RNA, 2256 nt, Homo sapiens
aggaaauaagagagaaaagaagaguaagaagaaauauaagagccaccauggcugaguggcuacucucggcuuccuggcaacgccgagcgaaagcuaugacugcggccgcggguucggcgggccgcgccgcggugcccuugcugcugugugcgcugcuggcgcccggcggcgcguacgugcucgacgacuccgacgggcugggccgggaguucgacggcaucggcgcggucagcggcggcggggcaaccucccgacuucuaguaaauuacccagagcccuaucguucucagauauuggauuaucucuuuaagccgaauuuuggugccucuuugcauauuuuaaaaguggaaauagguggugaugggcagacaacagacggcacugagcccucccacaugcauuaugcacuagaugagaauuauuuccgaggauacgaguggugguugaugaaagaagcuaagaagaggaaucccaauauuacacucauuggguugccauggucauucccuggauggcugggaaaagguuucgacuggccuuaugucaaucuucagcugacugccuauuaugucgugaccuggauugugggcgccaagcguuaccaugauuuggacauugauuauauuggaauuuggaaugagaggucauauaaugccaauuauauuaagauauuaagaaaaaugcugaauuaucaaggucuccagcgagugaaaaucauagcaagugauaaucucugggaguccaucucugcauccaugcuccuugaugccgaacucuucaaggugguugauguuauaggggcucauuauccuggaacccauucagcaaaagaugcaaaguugacugggaagaagcuuuggucuucugaagacuuuagcacuuuaaauagugacaugggugcaggcugcuggggucgcauuuuaaaucagaauuauaucaauggcuauaugacuuccacaaucgcauggaauuuaguggcuaguuacuaugaacaguugccuuaugggagaugcggguugaugacggcccaggagccauggagugggcacuacgugguagaaucuccugucuggguaucagcucauaccacucaguuuacucaaccuggcugguauuaccugaagacaguuggccauuuagagaaaggaggaagcuacguagcucugacugauggcuuagggaaccucaccaucaucauugaaaccaugagucauaaacauucuaagugcauacggccauuucuuccuuauuucaaugugucacaacaauuugccaccuuuguucuuaagggaucuuuuagugaaauaccagagcuacagguaugguauaccaaacuuggaaaaacauccgaaagauuucuuuuuaagcagcuggauucucuauggcuccuugacagcgauggcaguuucacacugagccugcaugaagaugagcuguucacacucaccacucucaccacuggucgcaaaggcagcuacccgcuuccuccaaaaucccagcccuucccaaguaccuauaaggaugauuucaauguugauuacccauuuuuuagugaagcuccaaacuuugcugaucaaacugguguauuugaauauuuuacaaauauugaagacccuggcgagcaucacuucacgcuacgccaaguucucaaccagagacccauuacaugggcugccgaugcauccaacacaaucaguauuauaggagacuacaacuggaccaaucugacuauaaagugugauguauacauagagaccccugacacaggagguguguucauugcaggaagaguaaauaaaggugguauuuugauuagaagugccagaggaauuuucuucuggauuuuugcaaauggaucuuacaggguuacaggugauuuagcuggauggauuauauaugcuuuaggacguguugaaguuacagcaaaaaaaugguauacacucacguuaacuauuaagggucauuucaccucuggcaugcugaaugacaagucucuguggacagacaucccugugaauuuuccaaagaauggcugggcugcaauuggaacucacuccuuugaauuugcacaguuugacaacuuucuuguggaagccacacgcuaagcggccgcuuaauuaagcugccuucugcggggcuugccuucuggccaugcccuucuucucucccuugcaccuguaccucuuggucuuugaauaaagccugaguaggaagucuagaguuuaaacauuuaaaucugcagaucccaauggcgcg